(19) United States
(12) Patent Application Publication
(10) Pub. No.: US 2025/0195723 A1
(43) Pub. Date: Jun. 19, 2025

(54) GRANULAR HYDROGEL BIOINKS WITH PRESERVED INTERCONNECTED POROSITY AND METHODS FOR MAKING AND USING THE SAME

(71) Applicant: The Penn State Research Foundation, University Park, PA (US)

(72) Inventors: Amir Sheikhi, University Park, PA (US); Mohammad Zaman Ataie, University Park, PA (US)

(21) Appl. No.: 18/848,709

(22) PCT Filed: Mar. 29, 2023
(86) PCT No.: PCT/US2023/016651

§ 371 Date: Sep. 19, 2024

Related U.S. Application Data
(60) Provisional application No. 63/324,774, filed on Mar. 29, 2022.
(60) Provisional application No. 63/367,521, filed on Jul. 1, 2022.
(60) Provisional application No. 63/424,286, filed on Nov. 10, 2022.

(51) Int. Cl. A61L 27/52; A61L 27/04; A61L 27/22; A61L 27/44; A61L 27/56

(52) U.S. Cl. CPC A61L 27/52; A61L 27/047; A61L 27/222; A61L 27/446; A61L 27/56; A61L 2400/06; A61L 2400/12

(57) ABSTRACT

Embodiments relate to granular hydrogel bioinks with preserved interconnected porosity for extrusion bioprinting and methods of use and making thereof. The granular hydrogel bioinks comprise hydrogel particles and materials/methods to reversibly connect them to each other without compromising void spaces. These materials/methods include but are not limited to nanoparticles, wherein the nanoparticles are absorbed onto the microgels and wherein the nanoparticles are configured to allow for electrostatic bonding of the microgels. These materials/methods can also include reversible functional groups, selected from guest-host groups and/or dynamic covalent bond forming groups.

### CROSS-REFERENCE TO RELATED APPLICATIONS

This patent application is related to and claims the benefit of priority of U.S. provisional application 63/324,774, filed on Mar. 29, 2022, the entire contents of which is incorporated by reference, and is further related to and claims the benefit of priority of U.S. provisional application 63/367,521, filed on Jul. 1, 2022, the entire contents of which is incorporated by reference, and is further related to and claims the benefit of priority of U.S. provisional application 63/424,286, filed on Nov. 10, 2022, the entire contents of which is incorporated by reference.

### FIELD OF THE INVENTION

Embodiments relate to granular hydrogel bioinks with preserved interconnected porosity for applications, such as extrusion bioprinting, and methods of use and making thereof.

### BACKGROUND OF THE INVENTION

Hydrogels have enabled the development of bioinks for tissue engineering and regeneration via providing physiochemically versatile tissue-mimetic microenvironments. Fabricated from polymer chains and/or colloidal particles, bulk hydrogels commonly attain nanoscale pores, limiting oxygen and metabolite diffusion and cell infiltration in thick three-dimensional (3D) constructs. As an example, a hydrogel scaffold fabricated from a purely elastic network of crosslinked polymers with a storage modulus G′ in the order of 10 kPa, mimicking soft tissues, will have pores in the order of ξ˜(G′/k_{B}T)^{-1/3}˜10 nm at physiological temperature. Here, k_{B} is the Boltzmann constant, and T denotes absolute temperature. Extensive efforts have been devoted to imparting microscale pores to hydrogels, most of which rely on non-bioorthogonal approaches, such as using porogens (e.g., salt crystals), ice-templating, or foaming. The main limitation of 3D bioprinting using conventional bulk hydrogel bioinks is the trade-off between shape fidelity and cell viability that is regulated by hydrogel stiffness/porosity.

According to the traditional “biofabrication window,” increasing the hydrogel stiffness, e.g., via increasing polymer concentration and/or crosslinking density, improves the construct shape fidelity while compromising the cell viability via reduced porosity.

To overcome the structural limitations of bulk hydrogels, microgels have been engineered as building blocks to assemble tissue engineering scaffolds, leveraging cell-scale interconnected microporosity and seamless cell infiltration. Granular hydrogel scaffolds have introduced a new biomaterial paradigm for tissue engineering, accelerating the regeneration of tissues, including skin, brain, and heart. These newly emerged scaffolds provide several unique advantages over bulk hydrogels, including (i) on-demand, in situ formation of microporous 3D constructs, (ii) pre-defined interconnected microporosity (regulated by microgel size and packing density), (iii) decoupled stiffness from porosity, and (iv) modular microarchitecture. Despite the recent advances in engineering microgel-based granular hydrogels for tissue engineering, additive manufacturing the granular hydrogels is not as trivial as bulk hydrogels. Microgels fabricated from various biomaterials, such as polyethylene glycol, chitosan, alginate, gelatin, and acrylic polymers, have been explored in bioinks, spheroid fabrication, and stimuli-responsive constructs. While bulk hydrogels may readily be rendered shear-thinning for extrusion-based bioprinting, granular hydrogels attain such rheological behavior only when they are in a “jammed” (tightly packed) state or embedded in a viscous matrix, which in turn compromises the void space among the microgels. Using large nozzles may improve the extrudability of granular hydrogels; however, achieving sub-mm resolution remains a bottleneck in the 3D bioprinting of granular microporous hydrogels.

Jamming in granular systems is defined as a physical process by which the viscosity increases as the volume fraction (φ) or particle number density increases. In other words, jamming signifies the transition of particulate systems from fluid-like to solid-like states, which varies in frictionless or frictional soft granular materials. In frictionless granules, jamming occurs beyond the state wherein particles are closely packed (Pc), i.e., ranging from 0.56 (random close-packed) to 0.74 (hexagonal close-packed) for monodispersed spheres. The jamming point is more complicated for frictional granules in which volume fraction is not the only indicator of jamming, and the jammed state may be reached at lower φ. Interaction between frictional microgels is an unexplored area in tuning the jamming, extrudability, and 3D bioprinting of granular systems. Confined microscale granules often undergo weak bonding among the adjacent particles via hydrogen bonding and/or electrostatic attraction. Such weak interactions enable the collective motion of microgels upon the exertion of sufficient pressure, enabling cohesive material flow through nozzles, followed by recovery after the backpressure is released. The microgels have to be jammed to maximize such weak interactions for enhanced printability, which inevitably minimize the void space among them.

Accordingly, there is currently an unmet need to develop granular bioinks that can preserve the microscale pores among microgels during and after extrusion bioprinting.

### BRIEF SUMMARY OF THE INVENTION

A generalized class of granular bioinks with remarkable extrusion printability, printing resolution, shape fidelity, and preserved interconnected microporosity have been produced. The microgel-microgel interactions may be engineered via the reversible interfacial self-assembly of heterogeneously charged nanoparticles. Embodiments may be based on the premise that dangling chains protruding out of polymeric microgels may adsorb the nanoparticles, followed by the charge-driven self-assembly of nanoparticles, imparting dynamic bonding to loosely packed microgels. Such dynamic bonds may form/break upon release/exertion of shear force, enabling the 3D printability of microgel suspensions without densely packing them. Embodiments may provide new opportunities for 3D bioprinting of tissues beyond the traditional biofabrication window.

In an exemplary embodiment, a method of forming a three-dimensional hydrogel scaffold comprises providing a granular bioink material comprising hydrogel particles, wherein the hydrogel particles are reversibly connected using a connection medium; extruding the bioink material; and exposing the extruded bioink material to a stimulus to form the three-dimensional hydrogel scaffold.

In some embodiments, the connection medium is nanoparticles.

In some embodiments, the hydrogel particles are selected from the group consisting of synthetic polymers, natural polymers, and/or semi natural polymers. In some embodiments, the hydrogel particles are gelatin methacryloyl.

In some embodiments, the step of providing the granular bioink material comprises forming gelatin methacryloyl by combining gelatin and methacrylic anhydride; forming the hydrogel particles by combining the gelatin methacryloyl and a photoinitiator; and combining the hydrogel particles with a connection medium, wherein the connection medium is nanoparticles.

In some embodiments, the photoinitiator is selected from the group consisting of lithium phenyl-2,4,6-trimethylbenzoylphosphinate, Ruthenium, and Irgacure 2959.

In some embodiments, the three-dimensional hydrogel scaffold comprises granular filaments with a diameter less than 400 μm.

In some embodiments, the stimulus is a light source with a wavelength of 250-785 nm. In some embodiments, the stimulus is a light source with a wavelength of 395-405 nm.

In some embodiments, the granular bioink material comprises silicate nanoparticles.

In an exemplary embodiment, a granular bioink material suitable for providing a three-dimensional hydrogel scaffold comprises gelatin methacryloyl hydrogel particles and a connection medium configured to reversibly connect the hydrogel particles.

In some embodiments, the connection medium is nanoparticles configured to be absorbed onto the hydrogel particles and wherein the nanoparticles are configured to allow for electrostatic bonding of the hydrogel particles.

In some embodiments, the nanoparticles are silicate nanoparticles.

In some embodiments, the nanoparticles are heterogeneously charged.

In some embodiments, the hydrogel particles have a size between 1 nm-1 mm.

In some embodiments, the hydrogel particles have an aspect ratio between 1-10.

In an exemplary embodiment, a three-dimensional hydrogel scaffold comprises a plurality of granular filaments, each filament comprising gelatin methacryloyl hydrogel particles and optionally nanoparticles.

In some embodiments, the filaments have a diameter less than 400 μm.

In some embodiments, the scaffold has a mean pore size of 1-1000 μm.

In some embodiments, the scaffold has a mean pore size of 10-100 μm.

In some embodiments, the scaffold has a minimum pore size of 100 nm.

In some embodiments, the scaffold has a minimum pore size of 10 μm.

In some embodiments, the scaffold has a void fraction of 10-40%.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 shows an exemplary method for forming an embodiment of the scaffold.

FIG. 2A shows a high-throughput step-emulsification microfluidic device.

FIG. 2B shows heterogeneously charged silicate nanoparticles that readily adsorb onto GelMA microgels, reversibly self-assemble at the interface, and yield shear-thinning nanoengineered granular bioink (NGB).

FIG. 2C shows extrusion and bioprinting of NGB, followed by photocrosslinking (scale bars are 3 mm and 400 μm).

FIG. 2D shows a visual comparison of filamentous extrusion using NGB, tightly packed, and loosely packed granular hydrogels.

FIG. 2E shows long hanging filaments formed from NGB (filament length L_{f}=45.0=5.0 mm, n=10), whereas tightly packed or loosely packed bioinks can only provide short (L_{f}=19.3+0.7 mm, n=10) or droplet-size (L_{f}=5.7±0.7 mm, n=10) filaments, respectively.

FIG. 2F shows radar plots of a qualitative comparison between the key properties of NGB, tightly packed, and loosely packed granular hydrogel bioinks as well as conventional bulk hydrogels.

FIG. 2G shows time-change of storage and loss moduli for the freshly prepared NGB, showing the rapid formation of a gel within ˜2 h as a result of nanoparticle self-assembly at microgel-microgel interfaces.

FIG. 2H shows viscosity of NGB versus steady shear rate.

FIG. 2I shows oscillatory strain (amplitude) sweep, furnishing the linear viscoelastic region (LVR) and the flow point of NGB.

FIG. 2J shows that NGB subject to alternating low (1%, i.e., lower than the flow point) and high (500%, i.e., higher than the flow point) strains at constant frequency (1 rad s⁻¹) undergoes complete recovery.

FIG. 3 shows a schematic of GelMA synthesis.

FIG. 4 shows a step-by-step preparation of loosely packed, tightly packed, and NGB systems.

FIG. 5 shows biofabrication windows for conventional bulk and granular hydrogels.

FIG. 6 shows silicate nanoparticle adsorption to 450 mg of microgel suspension (microgel average diameter 900 is 80±3 μm).

FIG. 7 shows frequency sweep at the constant strain of 0.1%, yielding the storage and loss moduli of NGB at frequency of 10⁻² to 10² rad s⁻¹ in the linear viscoelastic region.

FIG. 8A shows the transport of a high-molecular weight fluorescent dye (FITC-dextran, Mw=2 MDa, average Stokes radius˜27 nm) in different scaffolds with nano-/microscale pores.

FIG. 8B shows top and 3D orthogonal views of loosely packed microgel scaffold and 3D bioprinted NGB.

FIG. 8C shows raw fluorescence microscopy slices of loosely packed microgel scaffold and 3D bioprinted NGB analyzed using a custom-developed MATLAB script to obtain pore size distribution and void fraction (scale bar is 200 μm).

FIG. 8D shows void fraction and median pore diameter of loosely packed microgel and 3D bioprinted NGB scaffolds (NS: non-significant)

FIG. 9 shows the PDMS device used to conduct the FITC-dextran transport into scaffolds (scale bar is 10 mm).

FIG. 10A shows tapered nozzles with openings of 410, 250, and 200 μm (inner diameters, ID), corresponding to blue (22G), red (25G), and clear (27G) nozzles, respectively.

FIG. 10B shows a qualitative evaluation diagram of NGB 3D bioprinting using the blue (22G) nozzle.

FIG. 10C shows a qualitative evaluation diagram of NGB 3D bioprinting using the red (25G) nozzle.

FIG. 10D shows a qualitative evaluation diagram of NGB 3D bioprinting using the clear (27G) nozzle.

FIG. 10E shows an image of deposited NGB filaments via varying printing (nozzle movement) speed and extrusion pressure using the blue (22G) nozzle (scale bar is10 mm).

FIG. 10F shows an image of deposited NGB filaments via varying printing (nozzle movement) speed and extrusion pressure using the red (25G) nozzle (scale bar is10 mm).

FIG. 10G shows an image of deposited NGB filaments via varying printing (nozzle movement) speed and extrusion pressure using the clear (27G) nozzle (scale bar is10 mm).

FIG. 10H shows filament diameter versus printing speed for the blue (22G) nozzle at varying extrusion pressure.

FIG. 10I shows filament diameter versus printing speed for the red (25G) nozzle at varying extrusion pressure.

FIG. 10J shows filament diameter versus printing speed for the clear (27G) nozzle at varying extrusion pressure.

FIG. 11A shows evaluation of NGB extrusion 3D bioprintability based on varying printability window sizes (scale bar is 1 mm).

FIG. 11B shows merged tile scan microscopic images of the blue (22G) nozzle (scale bar is 4 mm).

FIG. 11C shows printability index for varying printing windows based on the blue (22G) nozzle.

FIG. 11D shows merged tile scan microscopic images of red (25G) nozzle (scale bar is 4 mm).

FIG. 11E shows printability index for varying printing windows based on the red (25G) nozzle.

FIG. 11F shows merged tile scan microscopic images of the clear (27G) nozzle (scale bar is 4 mm).

FIG. 11G shows printability index for varying printing windows based on the clear (27G) nozzle.

FIG. 11H shows hollow cylinders with a diameter of 5 mm and height of 5 mm printed using nozzles, leading to varying layer number and filament width (scale bar is 5 mm).

FIG. 11I shows height maintenance for 5 mm high hollow cylinders as an indicator for shape fidelity.

FIG. 11J shows hollow cylinders with a diameter of 5 mm and height of 10 mm printed using nozzles, leading to varying layer number and filament width (scale bar is 5 mm).

FIG. 11K shows height maintenance for 10 mm high hollow cylinders as an indicator for shape fidelity.

FIG. 12 shows a schematic of printability index of different shapes.

FIG. 13 shows steps of measuring printability index using stitched tile scan microscopic images.

FIG. 14 shows the printability index of hypothetically 3D bioprinted microgels using different arrangements.

FIG. 15 shows NGB 3D bioprinted structures of a logo with fluorescent microgels (top), inverted pyramid (middle), and human ear (30% of actual size, bottom).

FIG. 16A shows 3D bioprinted and photocrosslinked cylindrical NGB scaffolds to chemically assemble microgels, followed by oscillatory rheology and mechanical compression assessments.

FIG. 16B shows compressive stress-strain curves for the 3D bioprinted NGB, molded NGB, and bulk GelMA hydrogel.

FIG. 16C shows compressive modulus of the scaffolds calculated based on the slope of stress-strain curve at ˜5-15% of strain.

FIG. 16D shows storage modulus of the scaffolds versus angular frequency at a constant oscillatory strain of 0.1%.

FIG. 16E shows the storage modulus values for each sample obtained at an angular frequency of 1 rad s⁻¹ and an oscillatory strain of 0.1%.

FIG. 16F shows loss modulus of the scaffolds measured at a constant oscillatory strain of 0.1%.

FIG. 16G shows loss tangent of the scaffolds at an oscillatory frequency of 1 rad s⁻¹ and strain of 0.1%.

FIG. 17 shows compression modulus of bulk GelMA hydrogels, which are first pre-cooled (physically crosslinked) and then underwent chemical photocrosslinking compared with never-cooled photocrosslinked GelMA scaffolds (10% w/v).

FIG. 18 shows amplitude sweep tests of bulk GelMA, molded NGB, and 3D bioprinted NGB scaffolds at a constant frequency of 1 rad s⁻¹ and varying strain rates ranging from 10⁻¹ to 10²%.

FIG. 19A shows schematics of NGB 3D bioprinting, photocrosslinking, and microgel assembly, followed by topical 3D cell seeding and culture.

FIG. 19B shows cross-sectional images of bulk, tightly packed, loosely packed, and NGB scaffolds after topical cell seeding and culture for ˜4 h (scale bar is 200 μm).

FIG. 19C shows two-color live/dead fluorescence cell viability assay was performed over time on the 3D bioprinted NGB scaffolds, indicating the live and dead cells in green and red colors, respectively (scale bar is 200 μm).

FIG. 19D shows cell viability percentage, showing to a high cell viability (>90%) in the 3D bioprinted NGB scaffolds.

FIG. 19E shows metabolic activity of topically seeded cells in the 3D bioprinted NGB scaffolds over time, analyzed using the PrestoBlue™ kit.

FIG. 20 shows top, side, and 3D orthogonal fluorescence images of 3D bioprinted NGB scaffolds topically seeded with NIH/3T3 fibroblast cells after 5 min.

FIGS. 21A-C show in situ fabrication of granular hydrogel scaffolds from thermoresistive GelMA microgels (scale bar is 200 μm).

FIGS. 22A-D show material and mechanical characterization of granular hydrogel scaffolds made from thermoresistive GelMA microgels.

FIGS. 23A-C show biocompatibility assessment of granular hydrogel scaffolds made from thermoresistive GelMA microgels.

### DETAILED DESCRIPTION OF THE INVENTION

The following description is of an embodiment presently contemplated for carrying out the present invention. This description is not to be taken in a limiting sense but is made merely for the purpose of describing the general principles and features of the present invention. The scope of the present invention should be determined with reference to the claims.

It is contemplated that the terms “microgel” and “hydrogel particles” are used interchangeably throughout the present disclosure.

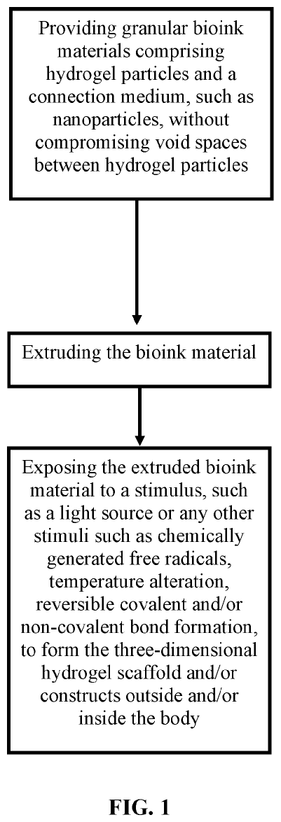

Referring to FIG. 1, embodiments may relate to a method of forming a three-dimensional hydrogel scaffold via a granular bioink material. The bioink material may comprise hydrogel particles and optionally a connection medium. The connection medium may be configured to reversibly connect the hydrogel particles. It is contemplated that the connection medium may be nanoparticles or any other medium, mechanism, method, etc. to reversibly connect the hydrogel particles.

It is contemplated that the hydrogel particles may be of any composition, including but not limited to gelatin methacryloyl, polyethylene glycol, hyaluronic acid, alginate, poly(N-isopropylacrylamide), polyacrylamide, or any combination thereof. In a preferred embodiment, the hydrogel particles are gelatin methacryloyl. It is further contemplated that the hydrogel particles may be of any shape, including but not limited to spherical, rod-like, disk-like, star-like, sheet-like, needle-like, irregularly shaped, porous, or any combination thereof. In a preferred embodiment, the hydrogel particles are spherical. It is further contemplated that the hydrogel particles may be of any size. In exemplary embodiments, the size of the hydrogel particles ranges from 1 nm-1 mm, preferably from 10-200 μm. It is further contemplated that the hydrogel particles may be of any aspect ratio. In exemplary embodiments, the aspect ratio of the hydrogel particles ranges from 0.01-100, preferably from 1-10.

In exemplary embodiments, the bio hydrogel particles may be formed using a photoinitiator. The photoinitiator may be selected from the group consisting of lithium phenyl-2,4,6-trimethylbenzoylphosphinate (LAP), Ruthenium, Irgacure 2959, or any other suitable photoinitiator.

In an embodiment wherein the hydrogel particles are gelatin methacryloyl, the gelatin methacryloyl may be formed by combining gelatin and methacrylic anhydride and optionally a photoinitiator. It is contemplated that any other synthetic, semi-synthetic/semi-natural, and natural polymer may be used to make the bioink.

Any microgel-microgel binding mechanism (e.g., connection medium) can be used to impart structural integrity to bioink before, during, or after 3D printing, e.g., nanoparticle self-assembly, photocrosslinking, dynamic covalent bond formation, any covalent bond formation, any physical interactions, such as ionic bonding and/or hydrogen bonding. In an embodiment wherein a connection medium is nanoparticles, it is contemplated that the nanoparticles may be absorbed onto the hydrogel particles and configured to allow for electrostatic bonding of the hydrogel particles. In particular, embodiments are based on the premise that dangling chains protruding out of the hydrogel particles may adsorb the nanoparticles, followed by the charge-driven self-assembly of nanoparticles, imparting dynamic bonding to loosely packed hydrogel particles. These dynamic bonds may form/break upon release/exertion of shear force, enabling the 3D printability of hydrogel suspensions without densely packing them.

In exemplary embodiments, the nanoparticles may be selected from the group consisting of silicate, clays, silica, mesoporous nanosilica, MXenes, and any other inorganic or organic particle that can induce reversible assembly of hydrogel particles. In a preferred embodiment, the nanoparticles are silicate nanoparticles. It is contemplated that the nanoparticles may be heterogeneously charged. In particular, the nanoparticles may be heterogeneous equipped with charged groups, which may readily absorb to a surface of the microgel and electrostatically bond to the charged groups on the exposed chains.

The method can involve extruding the granular bioink material, and exposing the extruded bioink material to a suitable stimulus to form the three-dimensional hydrogel scaffold. The stimulus may be selected from the group consisting of a light source, chemically generated free radical, temperature alteration, and reversible covalent and/or non-covalent bond formation. It is contemplated that the hydrogel scaffold may have favorable properties including, but not limited to, extrudability, printability, shape fidelity, and preserved interconnected porosity (e.g., ranging from 100 nm to 1000 μm, preferably 10-100 μm). In particular, it is contemplated that the hydrogel scaffold has a mean pore size of 1-1000 μm, preferably, 10-100 μm, a minimum pore size of 100 nm, preferably 10 μm, and a void fraction of 1-99%, preferably 10-40%.

### EXAMPLES

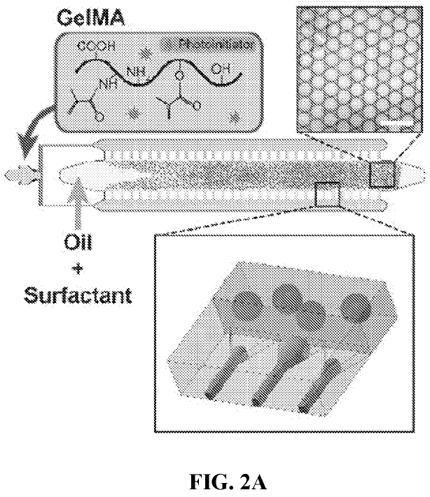

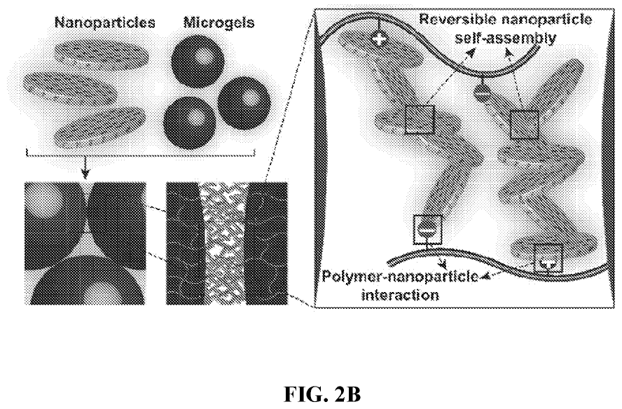

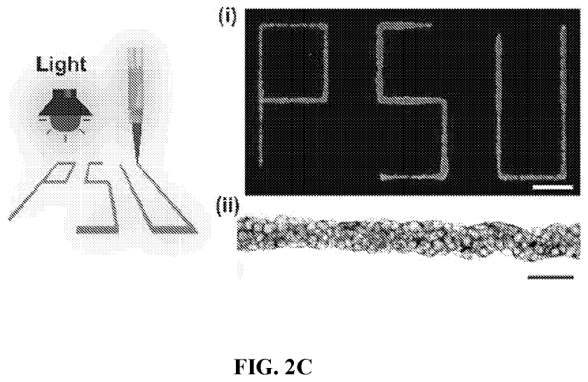

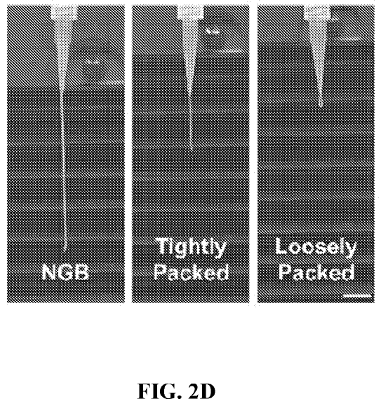

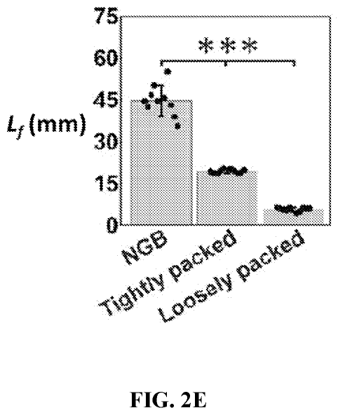

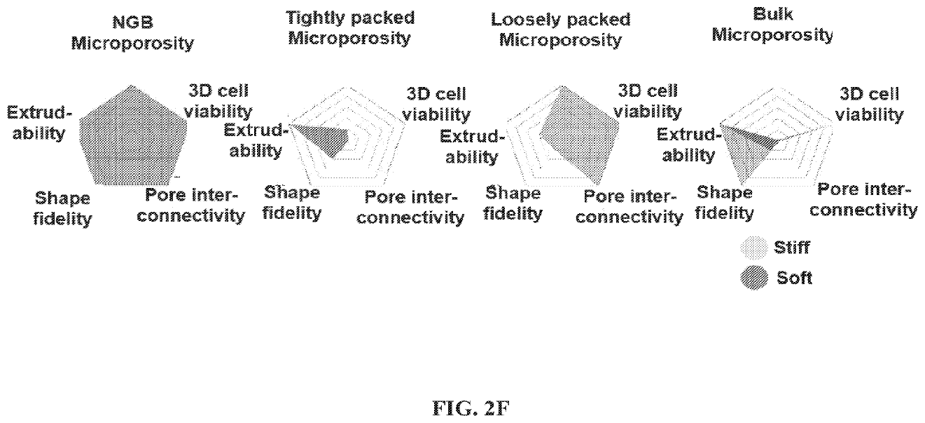

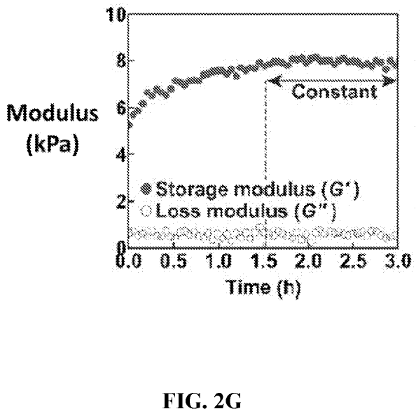

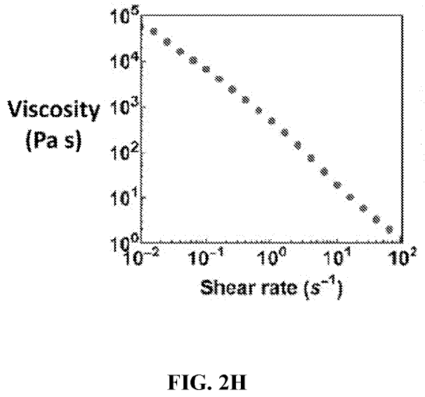

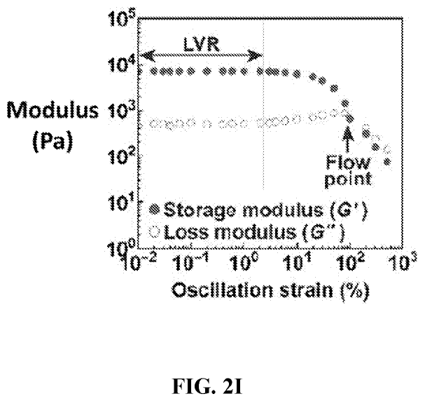

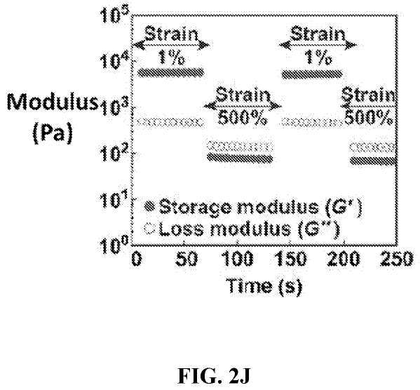

To generate hydrogel particles, the building blocks of NGB bioink, a high-throughput step emulsification microfluidic device was microfabricated to produce>40 million droplets per hour. FIG. 2A shows the device, containing highly parallelized steps (N=500) that generated surfactant-stabilized monodispersed droplets with an average diameter of 80±3 μm. Gelatin methacryloyl (GelMA), a modified protein-based biomaterial with an extracellular matrix (ECM)-mimetic chemical structure is used as the hydrogel particle precursor.

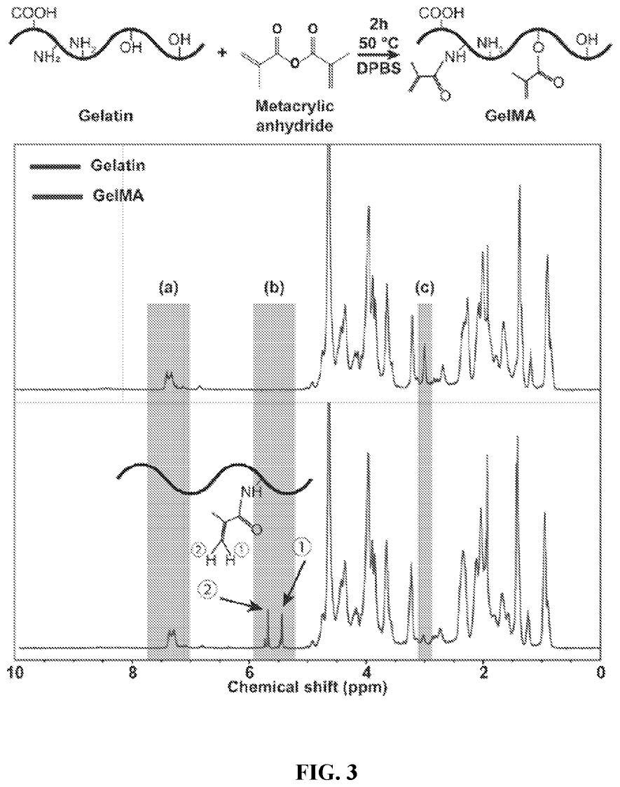

FIG. 3 shows a schematic of GelMA synthesis. Gelatin was mainly modified with methacryloyl groups (MA) via reacting with methacrylic anhydride at 50° C. in DPBS for 2 h. Proton nuclear magnetic resonance (¹HNMR) spectra of gelatin and GelMA (40 mg in D₂O) confirmed the MA modification of gelatin after the reaction. The peak for aromatic acids (a) in both gelatin and GelMA samples were considered as a reference. The functionalization was confirmed by the appearance of vinyl protons (b). The peak for lysine amino acid protons (c) was used for the calculation of MA degree of substitution DS=71±3% (n=3).

GelMA and a photoinitiator were used as the aqueous (dispersed) phase, expanding in a continuous oil phase to form photocrosslinkable GelMA droplets. The droplets were collected and stored at low temperature (4° C.) to form thermally crosslinked GelMA microgels, which were separated from the oil phase after destabilizing the emulsion. The NGB was then formed by imparting interfacial reversible microgel-microgel bonding using silicate nanoparticles to enable the coherent extrusion of granular bioink through nozzles while preserving the interstitial void spaces among the hydrogel particles (FIG. 2B). The nanoparticles were heterogeneously decorated with charged groups (anionic groups on the surface and cationic on the rim), which readily adsorbed to the hydrogel particle surface and electrostatically bonded to charged functional groups on the exposed (dangling) polymeric GelMA chains (carboxylates and primary amines), as schematically shown in FIG. 2B. The NGB was extrudable and photocrosslinked after printing using a light source (e.g., wavelength=395-405 nm, intensity=15 mW cm⁻²), yielding covalent bonds between the hydrogel particles, as presented in FIG. 2C, to form a stable construct with granular filaments of diameter<400 μm.

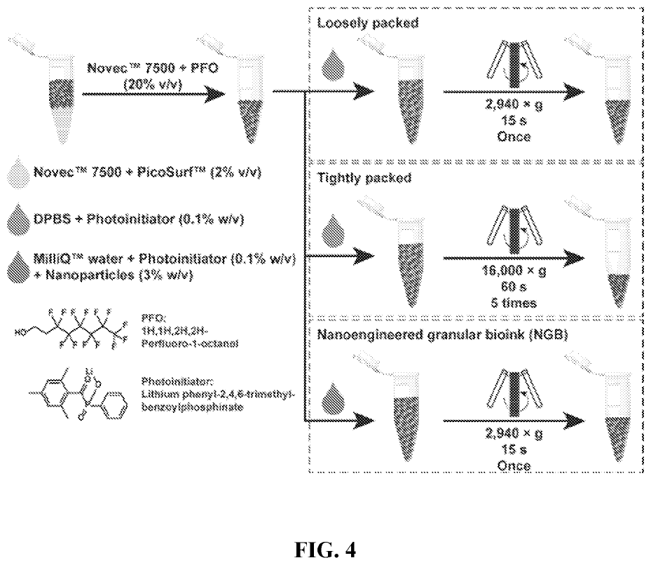

Hydrogel particles may readily be used to fabricate a variety of granular biomaterials with different extents of packing. To compare the NGB with commonly available granular systems, tightly or loosely packed hydrogel particle systems were fabricated by sequential centrifugation, as presented in FIG. 4.

FIG. 4 shows step-by-step preparation of loosely packed, tightly packed, and nanoengineered granular bioink (NGB) systems. After collecting the hydrogel suspension in oil from microfluidic device, the surfactant was removed using 1H, 1H,2H,2H-perfluoro-1-octanol (PFO) with a concentration of 20% v/v in Novec™ 7500 engineering oil. Loosely packed hydrogel suspensions were centrifuged at 2,940×g once, while tightly packed hydrogel suspension were centrifuged at 16,000×g five time, and the supernatant was removed afterwards. The NGB scaffolds were prepared similar to the loosely packed inks, in the presence of heterogeneously charged silicate nanoparticles.

Jamming has recently been used to increase the contact area among hydrogel particles and enhance their extrudability/printability. However, maximizing the contact area of hydrogel particles results in minimizing the interstitial void spaces among them. Therefore, a tradeoff between extrudability/printability and pore availability arises, implying that to achieve decent printability, interconnected microscale pores are compromised.

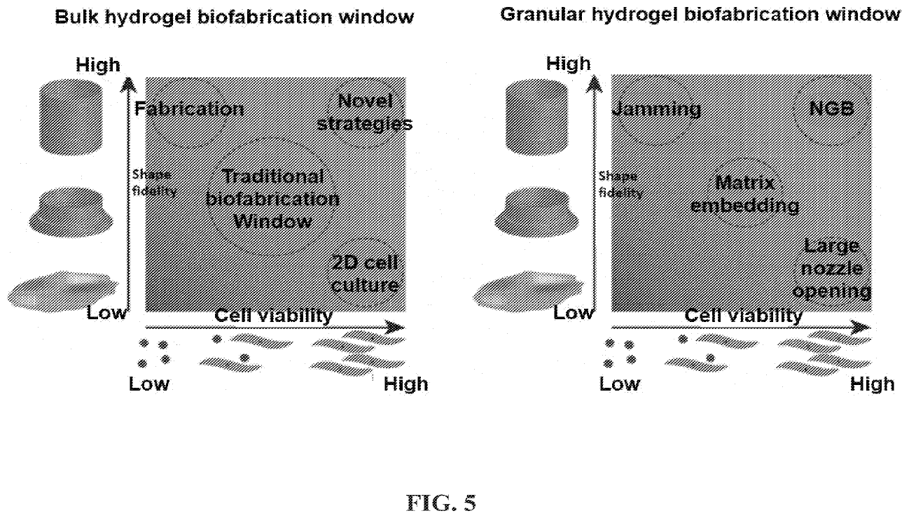

FIG. 5 shows such tradeoff within the realm of the biofabrication window of granular hydrogel bioinks. The NGB aims to addresses this persistent bottleneck in the 3D bioprinting of granular hydrogels. As seen in FIG. 5, for bulk hydrogels, the trade-off between shape fidelity and cell viability was governed by the polymer stiffness, crosslinking density, and/or concentration. Using granular hydrogel may enable 3D bioprinting beyond the conventional biofabrication window because the interstitial void spaces can be tailored regardless of individual hydrogel particle properties. So far, three different methods have been used for the 3D bioprinting of granular hydrogels: (i) tightly packing hydrogel particles, (ii) embedding hydrogel particles in a non-porous matrix, or (iii) using higher nozzle opening. Current granular hydrogel bioprinting methods share common shortcomings, including (i) the interconnected microporous structure is compromised, (ii) the printing resolution is in the mm scale, and/or (iii) multi-component materials are used as sacrificial gels for rendering the scaffold microporous.

FIG. 2D shows the filamentous extrusion of NGB compared with the tightly packed and loosely packed GelMA hydrogel particles using a nozzle with an inner diameter ID=410 μm. The length of hanging filaments (L_{f}) for each bioink is presented in FIG. 2E, showing that the extrusion of NGB through a nozzle generates longer hanging filaments in comparison with other tightly packed systems. Importantly, the loosely packed granular bioink was not able to yield any filaments via extrusion as a result of suboptimal interparticle interactions. Using smaller nozzle opening (ID=200 μm), the NGB and tightly packed bioinks were still extrudable; however, the loosely packed system could not be extruded, and sudden jetting due to nozzle cloggage was observed. Table 1 presents the other granular hydrogel bioinks that have been used as bioinks for extrusion-based bioprinting; however, none of these systems have been able to offer a ready-to-print single-component bioink with an interconnected microporous structure. In addition, efforts to extrude loosely packed hydrogel suspensions have resulted in no extrusion, nozzle cloggage, or jamming, depending on the nozzle size.

TABLE 1
Granular hydrogel bioinks

| | Hydrogel particle | | |
|---|---|---|---|
| | material | Printing methodology | Cell penetration studies |
| Embodiments | Gelatin | Nanoengineered | Yes. Immediate |
| described herein | methacryloyl | granular | penetration and cell |
| | | bioink | infiltration |
| [5] | Polyethylene glycol | Jamming (with | Not applicable |
| | diacrylate - | vacuum No filtration) | |
| | agarose | | |
| [6] | Polyethylene glycol | Using large nozzle | Not applicable |
| | | opening | |
| [7] | ChitoMA PVA | Jamming (micro- | No |
| | | flake centrifugation) | |
| [8] | Gelatin | Jamming | No penetration after |
| | methacryloyl and | | printing. Penetration |
| | gelatin (sacrificial | | observed after |
| | material) | | sacrificial gel removal |
| [9] | 2-acrylamido-2- | Jamming (with | No |
| | methylpropane | vacuum, no filtration) | |
| | sulfonic acid | | |
| [10] | Polyethylene glycol | Not mentioned | Not applicable |
| [11] | Polyethylene glycol | Jamming | No |
| [12] | Gelatin | Matrix embedding | No |
| | methacryloyl | (suing no bulk | |
| | | GelMA as matrix) | |
| [13] | Oxidized and | Jamming (micro- | No |
| | methacrylate | flake no | |
| | alginate | centrifugation) | |
| [14] | Gelatin (as | Matrix embedding | Not applicable |
| | sacrificial gel) | (using different types | |
| | | of bulk matrices) | |

FIG. 2F shows radar plots of qualitative comparison between the key properties of NGB, tightly packed, and loosely packed granular hydrogel bioinks as well as conventional bulk hydrogels. The unique advantages of NGB over all other granular bioinks as well as conventional bulk hydrogels for extrusion bioprinting are qualitatively presented in FIG. 2F. Notably, cell-size interconnected microporosity, extrudability, and/or 3D cell viability was compromised using other types of bioinks. Jammed platforms maximized the microgel-microgel contact area, resulting in minimized pore size and interconnection, which may diminish cell viability. Loosely packed systems maintained their interconnected microporosity but are non-trivial to extrude/bioprint. Conventional bulk hydrogels are typically extrudable, but their pores are about three orders of magnitude smaller than typical cell size, and their stiffness dictates the tradeoff between shape fidelity and cell viability, as described in the bulk hydrogel conventional biofabrication window (see FIG. 5).

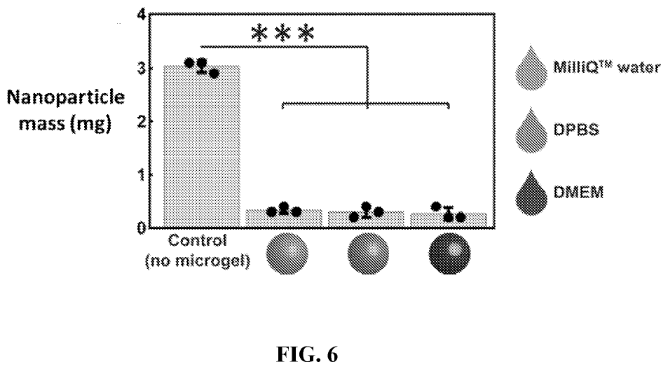

Rheological behavior of bioinks regulate their extrudability, printability, and shape fidelity in extrusion-based bioprinting. The NGB was characterized using different rheological assessments to confirm its suitability for extrusion bioprinting. FIG. 6 shows silicate nanoparticle adsorption to 450 mg of microgel suspension (hydrogel particle average diameter is 80±3 μm). Hydrogel particles were made of ultra-pure Milli-Q® water, DPB'S, or DMEM. Results showed that almost all the nanoparticles may be adsorbed to the hydrogel particles. The nanoparticles are adsorbed onto the hydrogel particles (see FIG. 6), followed by time-dependent self-assembly. To confirm the self-induced assembly in the NGB, low-amplitude oscillatory rheology at constant strain of 0.1% and frequency of 1 rad s⁻¹ is performed on freshly prepared NGB, yielding the evolution of storage (G′) and loss (G″) moduli, as presented in FIG. 2G. Storage modulus reached a plateau in about 2 h (average time=1.85±0.32 h, average storage modulus=7.82±1.52 kPa, n=3). Being a shear-thinning material is considered as a prerequisite for extrusion-based bioprinting. In FIG. 2H, viscosity was measured at varying shear rates via a shear ramp test, showing that the NGB has a shear-thinning rheological behavior, i.e., its viscosity decreases as shear rate increases. Jammed granular hydrogels have also been reported to be shear-thinning; however, the effect of wall shear which is applied by the nozzle in a confined area (e.g., nozzle orifice) has not been considered in the rheological assessments.

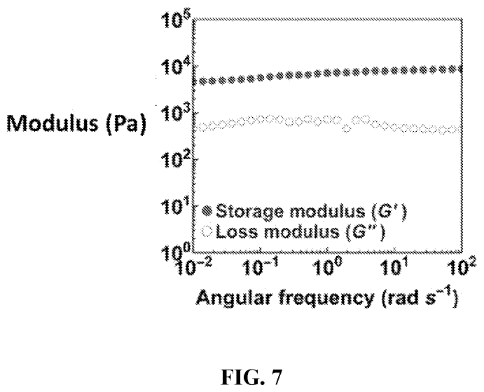

FIG. 2I presents the oscillatory amplitude (strain) sweep test performed on the NGB at a constant, low frequency (1 rad s⁻¹) yielding the linear viscoelastic region (LVR, at strain<10%) and flow point (strain˜100%). In addition, the frequency sweep test was conducted at a constant, low strain (0.1%) in the LVR region (see FIG. 7) showing almost constant storage and loss moduli within the range of 0.01 to 100 rad s⁻¹. Applied shear should be higher than the flow point to enable the extrusion of NGB. In addition, the bioink must rapidly recover its solid-like behavior after extrusion to form constructs with high shape fidelity. FIG. 2J shows the recovery test, which was performed via alternating strains over time at low (1%, lower than flow point) and high (500%, higher than flow point) values to ensure that the filaments promptly recovered their solid-like structure after the extrusion. At the low strain, the NGB had a gel-like behavior (G′>G″), which was reversibly altered to a liquid-like behavior (G″>G′) at the high strain.

The extrusion bioprinting of granular hydrogels was regulated by the microgel-microgel interactions during and after extrusion. Such interactions were maximized in jammed and matrix-embedded granules; however, maximizing the contact area of hydrogel particles or embedding them in a non-porous matrix compromised the microporosity. Surface-nanoengineered loosely packed hydrogel particles using shear-responsive nanoparticles that can reversibly self-assemble may enable strong microgel-microgel interactions, facilitating extrusion via micro-nozzles while preserving interconnected microscale pores among the hydrogel particles.

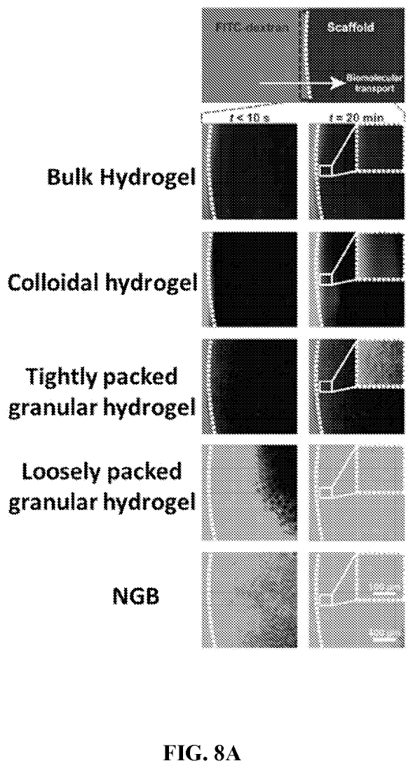

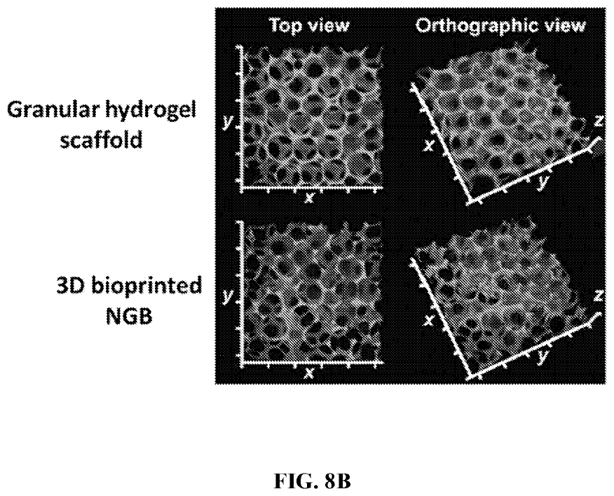

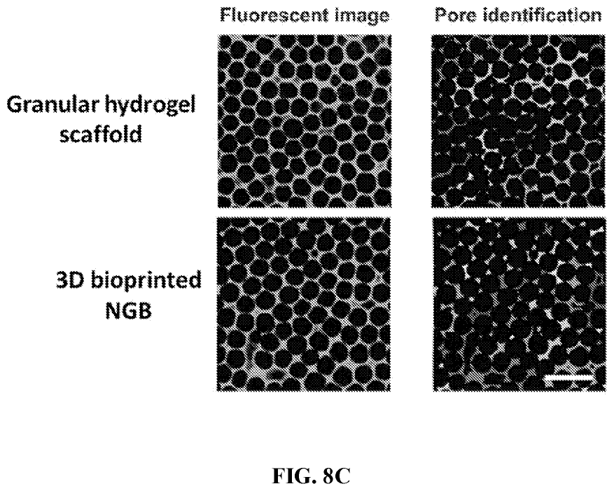

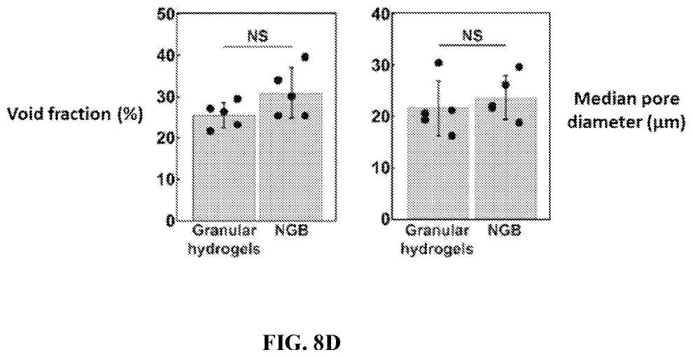

To test the hypothesis, molecular transport in different nano-/microporous scaffolds was assessed in FIGS. 8A-D. As shown in FIG. 8A, the transport of a high-molecular weight fluorescently-labeled polysaccharide (FITC-dextran, average molecular weight=2 MDa, Stokes radius=27 nm) in nano-/microporous scaffolds. As shown in FIG. 8A, nanoporous scaffolds, such as bulk GelMA and silicate colloidal hydrogels, as well as the densely packed granular hydrogel did not permit significant dye penetration within 20 min. Loosely packed granular hydrogel systems and NGB permitted immediate dye penetration within seconds.

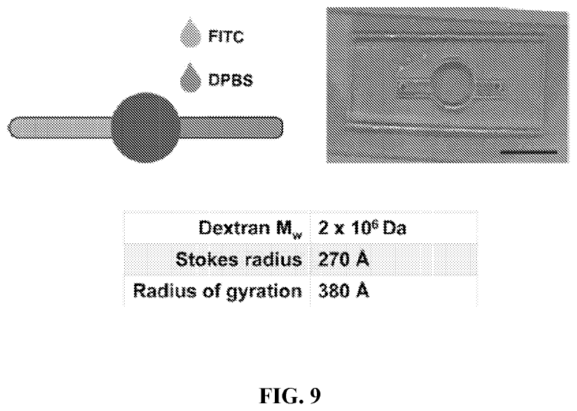

FIG. 9 shows the PDMS device used to conduct the FITC-dextran transport into scaffolds. The cylindrical well is 8 mm in diameter and 5 mm deep. Scaffolds (cylindrical with diameter of 8 mm and height of 3 mm) were placed in the well, exposed to FITC-dextran from one side and DPBS from the other side for visualizing the transport and prevent drying, respectively. The fluorescence images were acquired over time at a fixed location close to the contact point between the fluorescent dye and the scaffold using a low magnification objective (total magnification of 40×).

The scaffolds were exposed to FITC-dextran from the side (see FIG. 9), and sheet fluorescent images were acquired immediately after exposure (t<10 s) and after 20 min. The large FITC biomolecule was not able to diffuse in the nanoporous structures of bulk GelMA or nanosilicate colloidal hydrogels. The jammed microgel scaffold underwent negligible FITC penetration after 20 min. Importantly, the biomolecule was readily transported through the loosely packed microgel scaffolds and photocrosslinked NGB. The results imply that the NGB scaffolds attain microporosity as hypothesized.

To quantify the interconnected porosity of NGB scaffolds, they were labeled using the FITC-dextran and 3D imaged. FIG. 8B shows the top and orthogonal views of 3D void spaces among the microgels in the scaffolds, attesting to their interconnectivity. The fluorescent dye had a large hydrodynamic size, therefore it cannot penetrate in the hydrogel particles, labeling only the void spaces. Raw fluorescence microscopic slices were analyzed using a custom-developed MATLAB code to identify the pores, as presented in FIG. 8C. The area of pores for each 2D slice was measured, and the equivalent diameter was calculated accordingly. The median pore diameter and void fraction of microporous scaffolds are reported in FIG. 8D, indicating no significant difference between the NGB and loosely packed scaffolds.

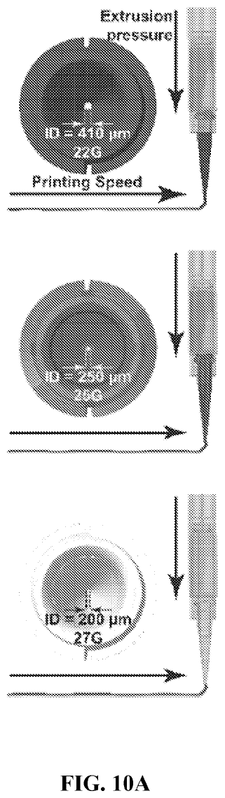

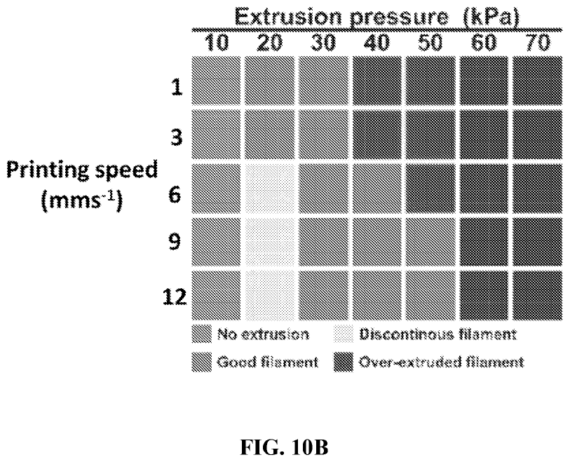

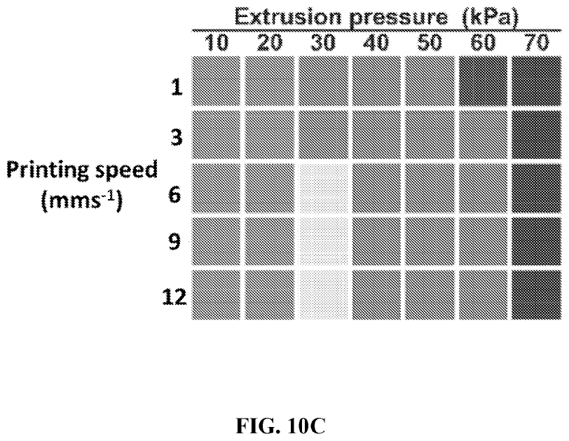

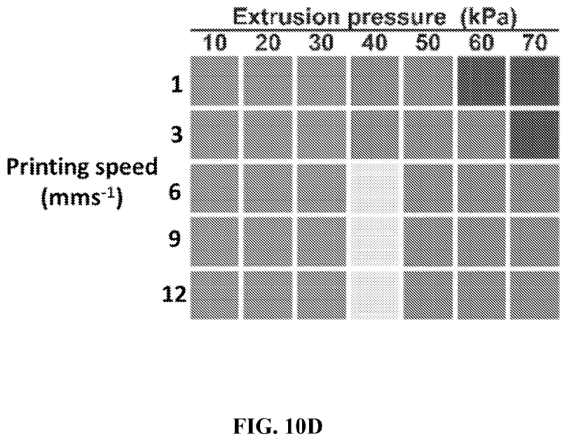

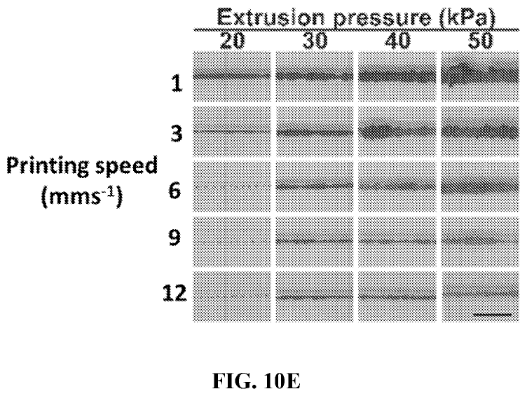

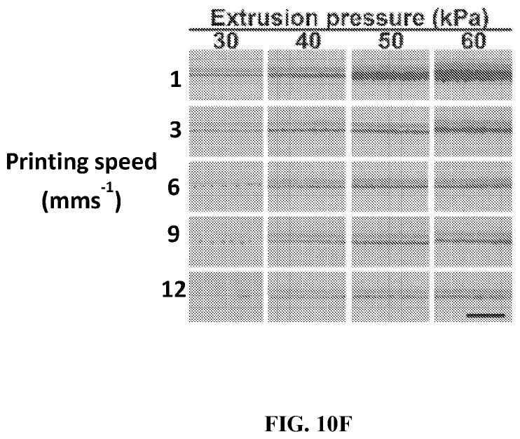

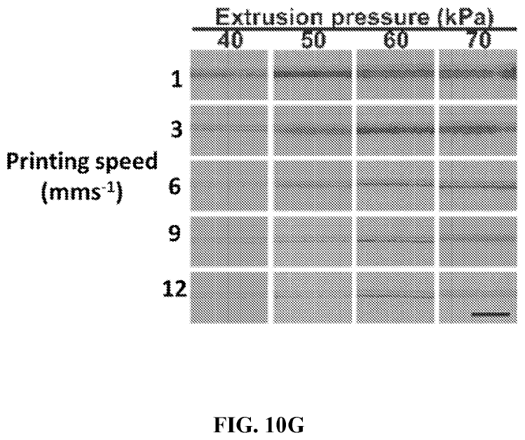

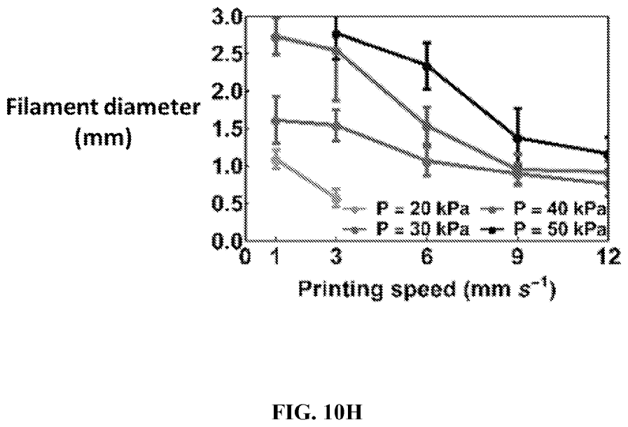

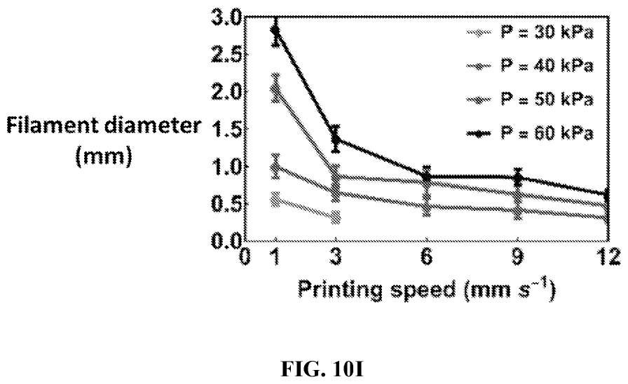

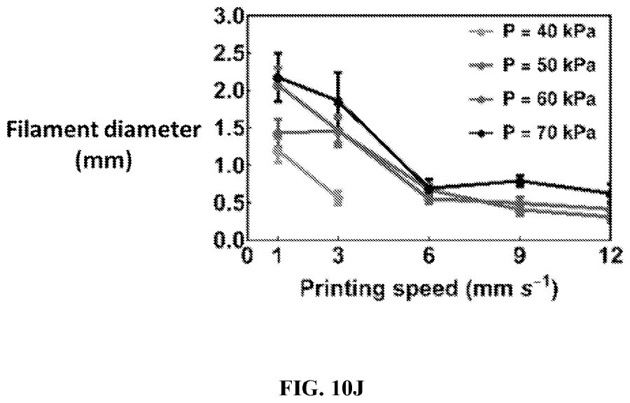

Standard tapered bioprinting micro-nozzles were used at varying conditions to optimize the NGB extrusion bioprinting at the sub-mm resolution. Three nozzle opening IDs of 410, 250, and 200 μm, represented by blue, red, and clear nozzles, respectively, were used (FIG. 10A), and the extrusion force was applied pneumatically to a piston, driving the airtight NGB through the nozzles to deposit it in the air on a planar substrate (printing bed). Qualitative and quantitative analyses of the deposited filaments at varying extrusion pressure and printing (nozzle movement) speed are presented in FIG. 10. The quality of NGB bioprinting at varying printing speed and extrusion pressure is presented in FIGS. 10B, 10C, and 10D, for the blue, red, and clear nozzles, respectively.

For each nozzle, there exists a minimum onset pressure to initiate the extrusion regardless of the printing speed. The smaller the nozzle opening, the higher the onset extrusion pressure. This is mainly a result of increased wall resistance in narrower channels. In a larger nozzle opening (i.e., blue nozzle, tip ID=410 μm, FIG. 10B), filaments are extruded with lower backpressure (i.e., 20 kPa); however, over-extrusion is observed at pressure≥60 kPa. Note that over-extrusion is directly affected by the printing height, defined as the distance between the nozzle tip and the printing bed, which is maintained constant at 400 μm for all the experiments. In 3D printing, height should be optimized based on the filament diameter and nozzle ID. Increasing or decreasing the printing height affects the final shape of extruded filament and shape fidelity. Constant printing height resulted in smashed-like filaments for blue (FIG. 10B) and red nozzle (FIG. 10C). As the nozzle opening decreases, the over-extrusion was observed at higher extrusion pressure (≥70 kPa for the red nozzle, tip ID=250 μm, FIG. 10C) or was never observed at up to 70 kPa for the clear nozzle (tip ID=200 μm, FIG. 10D).

Increasing the printing speed may result in discontinuous filaments at relatively low extrusion pressure. This condition is a result of a tradeoff between the amount of NGB dispensed from the tip and the contact time between the NGB and the substrate. As an example, for the blue (FIG. 10B), red (FIG. 10C), or clear (FIG. 10D) nozzles, the NGB was well extrudable at extrusion pressure of 20 kPa, 30 kPa, or 40 kPa, respectively, when the printing speed was lower than 6 mm s⁻¹. The images of extruded NGB filaments at varying extrusion pressure and printing speed are presented in FIGS. 10E, 10F, and 10G for the blue, red, and clear nozzles, respectively. The following universal trend was observed for all the nozzles: (i) thinner NGB filaments are deposited when the printing speed increases at a given extrusion pressure as a result of a lowered contact time between the NGB and the contact point; (ii) at a constant printing speed, increasing the extrusion pressure yields thicker filaments as a result of increased deposited NGB at the contact point; (iii) increasing the printing speed at a constant extrusion pressure will eventually result in the deposition of discontinuous filaments. Filament diameter is plotted versus printing speed in FIGS. 10H, 10I, and 10J for the blue, red, and clear nozzles, respectively. The minimum printing resolution for the blue, red, and clear nozzles were about 0.5, 0.3, and 0.2 mm, respectively, all slightly higher than the nozzle IDs. Maximum printed filament diameters were >3, 3, and 2 mm for the blue, red, and clear nozzles, respectively.

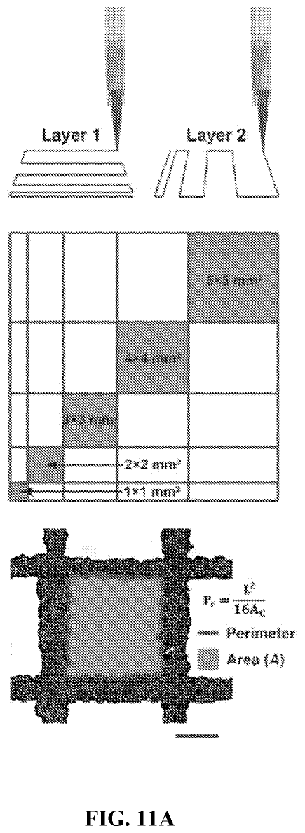

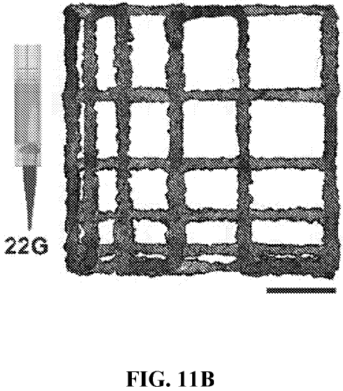

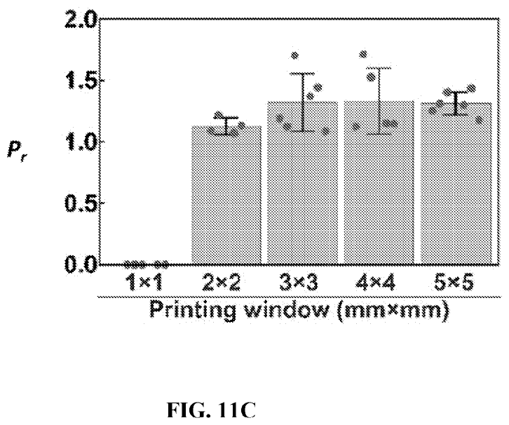

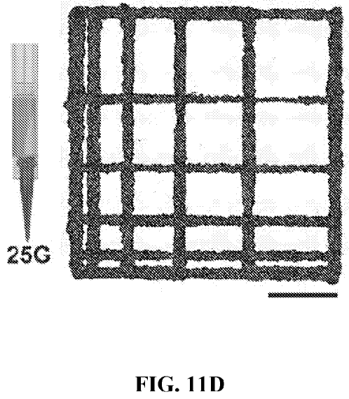

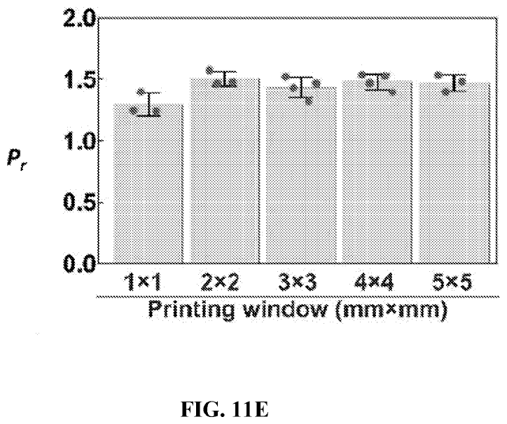

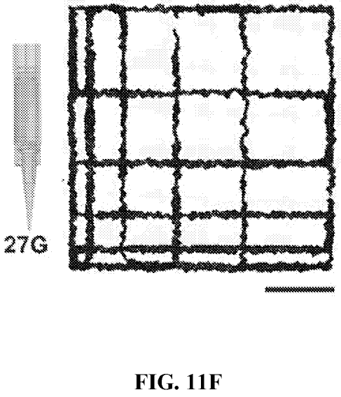

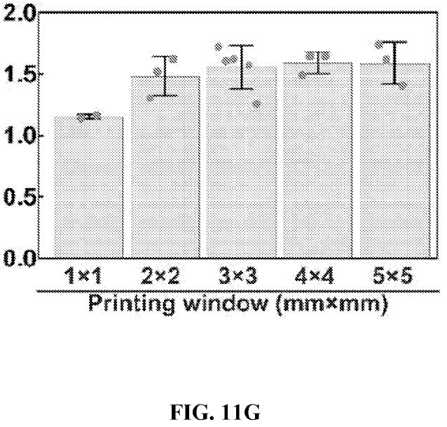

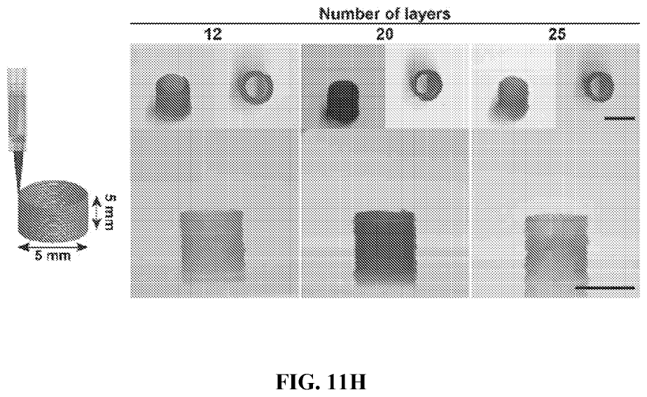

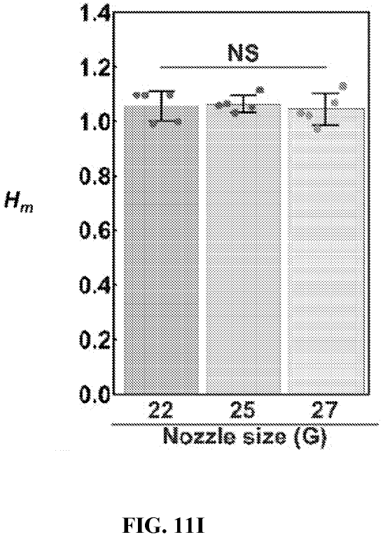

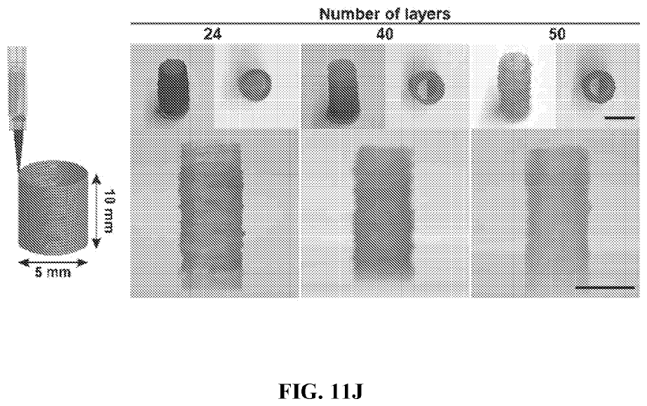

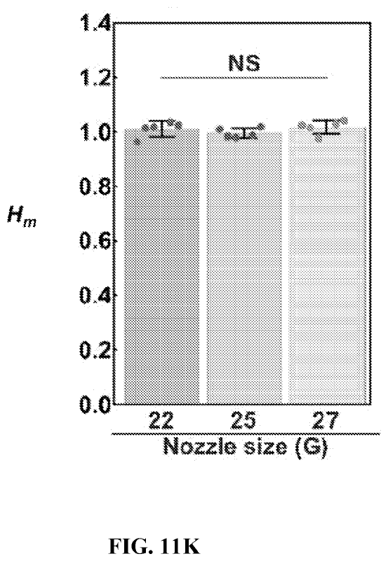

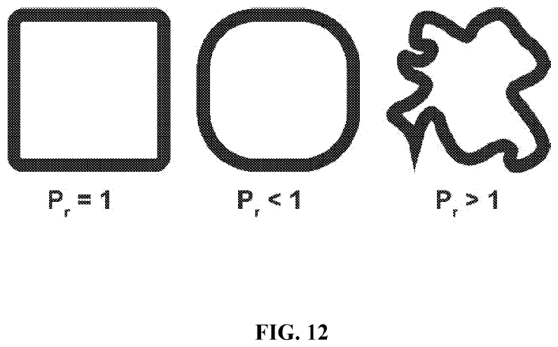

Standard printability and shape fidelity assays were performed based on established protocols. For printability analysis, horizontal (0-degree) and vertical (90-degree) layers were custom-coded and deposited in two consecutive layers on top of each other, as shown in FIG. 11A. These layers were printed with 15 mm length and 1-, 2-, 3-, 4-, and 5-mm offset (sub-panel i), yielding five squares emerging on the diagonal of printed structure, named printability windows (sub-panel ii). Printability was quantified based on the printability index (Pᵣ), defined as the ratio of actual area (A) to the perimeter (L) of each printability window (sub-panel iii). Pᵣ is a modified circularity formula to determine how the squares are deposited on the x-y plane. Accordingly, as presented in FIG. 12, Pᵣ=1, Pᵣ<1, and Pᵣ>1 correspond to perfect square, more circular, and more irregular-shaped printing windows, respectively.

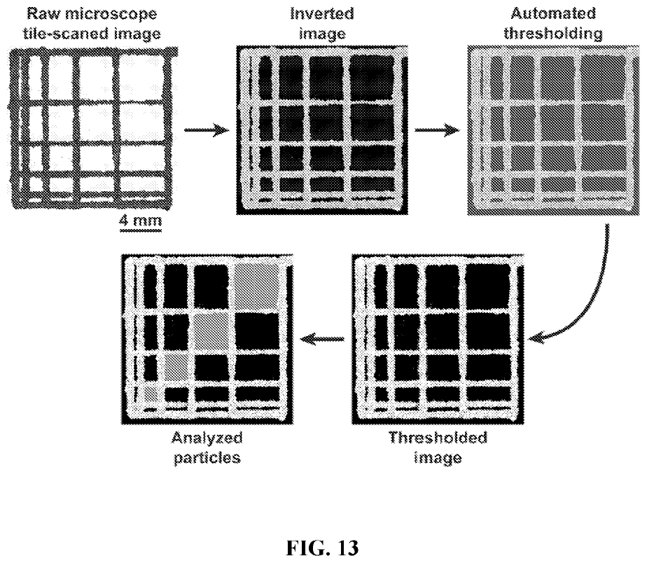

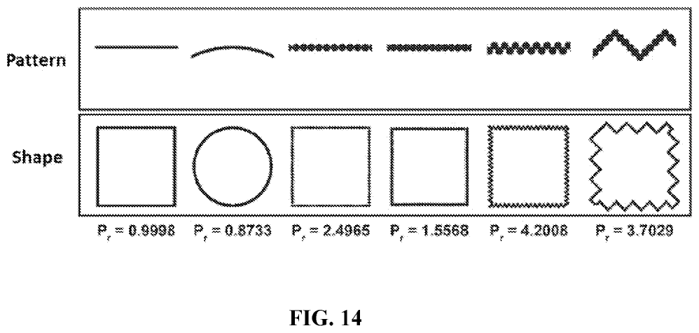

NGB printability indices were measured by varying the nozzle opening. The printed structures were imaged using merged tile-scanned microscopy for the blue (FIG. 11B), red (FIG. 11D), and clear (FIG. 11F) nozzles. The microscopic images were exported, inverted, and thresholded, followed by analyzing the printability windows to find the perimeter and area (FIG. 13). In general, granular hydrogel bioink printability windows are irregularly shaped (Pᵣ>1) due to their inherent granular building blocks. FIG. 14 shows a schematically drawn 2 mm×2 mm printability window with 80 μm circular building blocks, confirming Pᵣ>1 using different arrangements and shapes. As shown in FIG. 11C, the 1 mm×1 mm printability window could not be printed using the blue nozzle, and 2 mm×2 mm printability window was more irregularly shaped than the larger windows. This is a result of the higher thickness of deposited filament using the larger (blue) nozzle. The values of Pᵣ plateau at ˜1.5 for larger printability windows (larger than 3 mm×3 mm), attesting to well-defined squares. Using smaller nozzle openings, e.g., red and clear nozzles as shown in FIGS. 11E and 11G, respectively, the 1 mm×1 mm was printed; however, compare with the larger printability windows, the shape was more circular. Therefore, the printability of 1 mm×1 mm windows for red (Pᵣ˜1.3) and green (Pᵣ˜1.2) nozzles is lower than the Pᵣ for other printability windows (Pᵣ˜1.5). Overall, the NGB attains high printability depending on the micro-nozzle ID, which dictates the filament thickness.

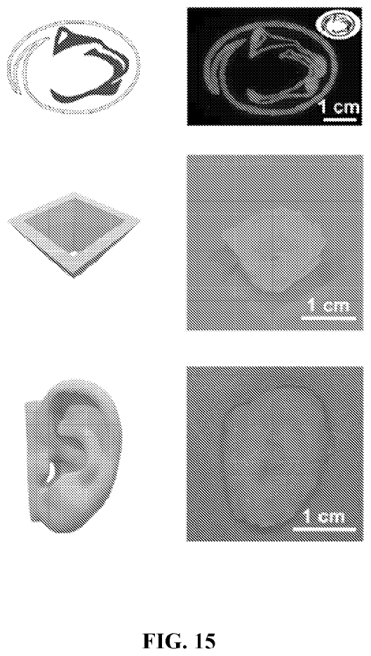

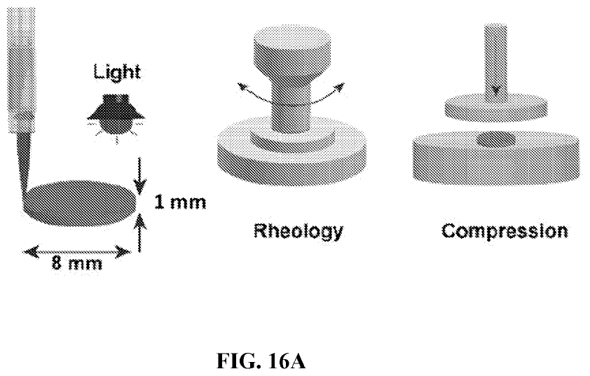

The relative evaluation of layer stacking in hollow cylinder models was performed to evaluate the shape fidelity. Cylinders with a base diameter and height of 5 mm were 3D bioprinted, as presented in FIG. 11H. The number of stacked layers was different depending on the filament length, dictated by the micro-nozzle IDs. For blue, red, and clear nozzles, the number of stacked layers were 12, 20, and 25, respectively, and the design (theoretical) layer height were 420, 250, and 200 μm, respectively. FIG. 11I shows the height maintenance factor (Hₘ), comparing the actual height (Hₐ) with the theoretical height (Hₜ). Hₘ was ˜1 for all the 3D bioprinted constructs, showing high shape fidelity. To further asses the shape fidelity, the height of printed cylinders was increased 2-fold while maintaining the base diameter unchanged, as shown in FIG. 11J. In this case, the number of stacked layers increase to 24, 40, and 50 layers for the blue, red, and clear nozzles, respectively. The values of Hₘ are presented in FIG. 11K, showing enhanced shape fidelity (Hₘ˜1) even in high aspect ratio constructs. The results show the high shape fidelity of 3D bioprinted NGB constructs. To further demonstrate the printability of NGB, complex shapes, such as the Penn State Nittany Lion logo, an inverted pyramid, and human ear models were printed, as shown in FIG. 15.

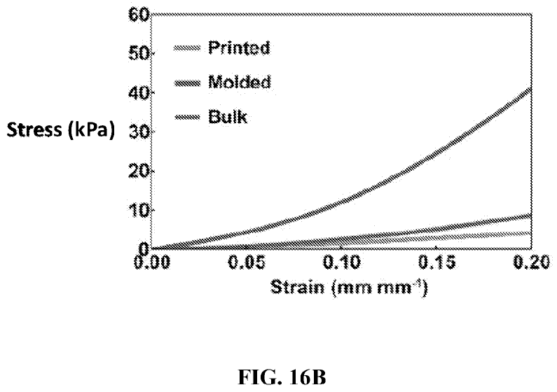

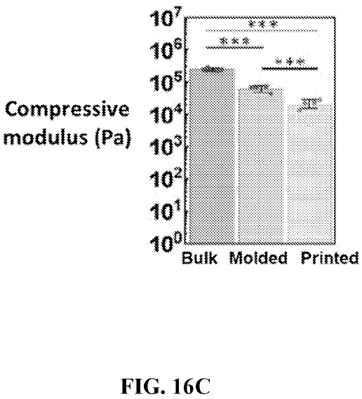

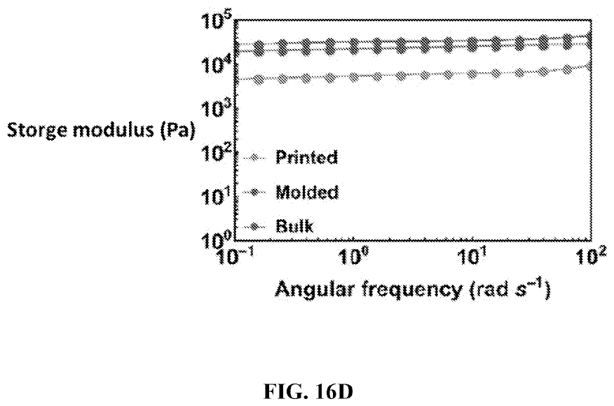

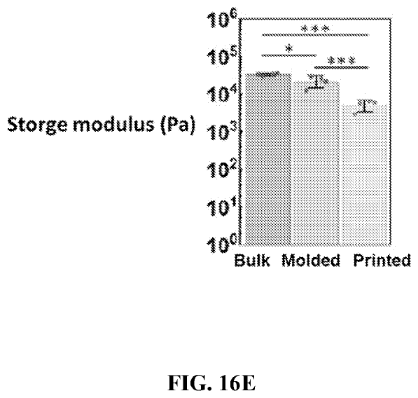

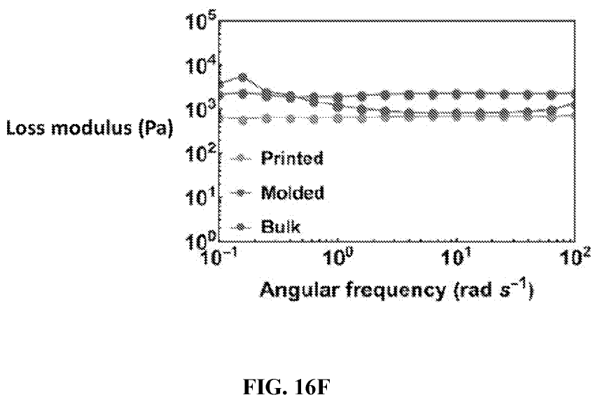

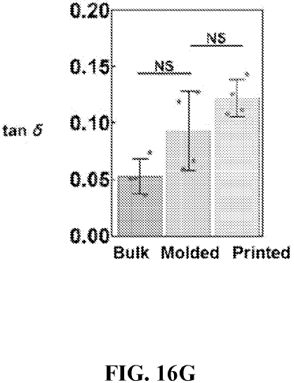

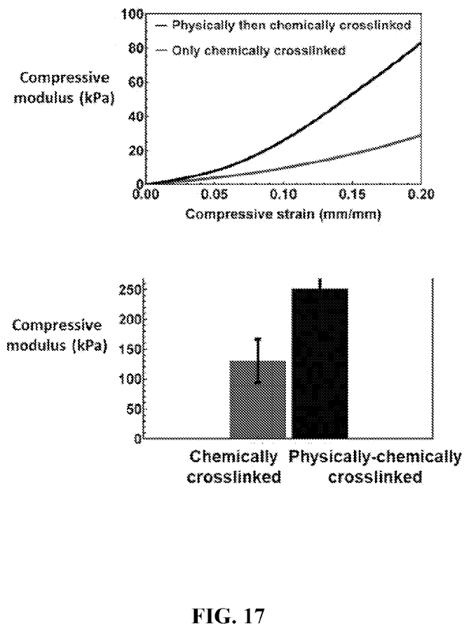

Mechanical and rheological properties of 3D bioprinted NGB scaffolds were evaluated using compression tests and oscillatory rheology, respectively. NGB scaffolds consisting of three bioprinted stacked layers were photocrosslinked using a light source (wavelength=395-405 nm, intensity=15 mW cm⁻²) (FIG. 16A). Molded bulk GelMA and NGB scaffolds were considered as controls. Compression tests were conducted using a mechanical tester to obtain the compressive stress versus strain, as presented in FIG. 16B. At any given strain below 20%, the compressive stress for the bulk hydrogel was higher than the granular scaffolds. The molded NGB had a higher compressive stress than the 3D bioprinted NGB scaffold. FIG. 16C presents the compressive modulus of 3D bioprinted NGB, molded NGB, and bulk scaffolds, calculated from the slope of elastic region in the stress-strain curves at ˜5-5% of strain. Bulk scaffolds had a significantly higher compressive modulus than granular platforms due to the lack of microscale porosity. Bulk and granular scaffolds were prepared similarly, meaning that a physical crosslinking step (i.e., partial gelation of protein-based biomaterial at 4° C.) was performed before photochemically crosslinking the hydrogels. Two-step crosslinking of bulk hydrogels showed higher compression modulus compared with only chemically crosslinked hydrogels, as shown in FIG. 17. In addition, 3D bioprinted NGB scaffolds had lower compressive modulus than the molded NGB, which was expected due to the presence of inter-layer and inter-filament voids and inadequate bonding. Similar behaviors have been reported for additive manufacturing of nanocomposites, thermoplastic polymers, biomaterials, and jammed granular hydrogels. Overall, the high robustness of 3D bioprinted NGB scaffolds, with a compressive modulus in the order of 10 kPa, rendered them easy to handle and manipulate.

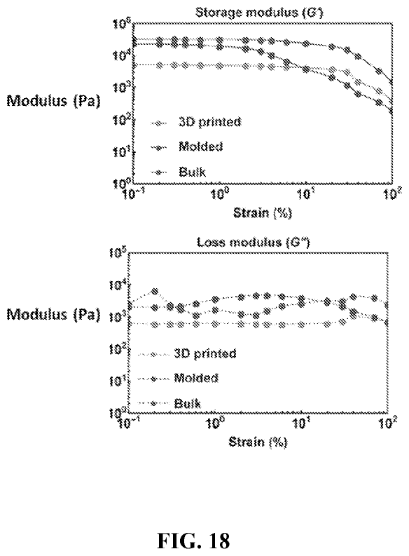

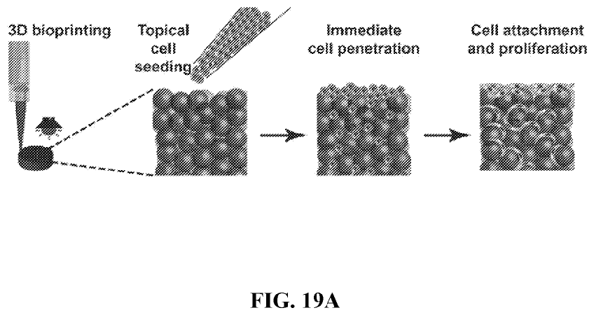

The viscoelastic properties of 3D bioprinted NGB, molded NGB, and bulk GelMA scaffolds were analyzed using oscillatory rheology. First, the scaffolds underwent oscillatory amplitude sweep at a constant frequency (1 rad s⁻¹) to determine the LVR, as presented in FIG. 18. Then, frequency sweep from 0.1 to 100 rad s⁻¹ was performed at a low oscillatory strain of 0.1% to obtain the storage (FIG. 16D) and loss (FIG. 16F) moduli at the LVR. Storage moduli of different scaffolds show solid-like behavior without any significant changes within the investigated frequency range. FIG. 16E presents the comparative analysis of storage modulus among the scaffolds at 0.1% of strain and 1 rad/s of frequency, which shows a similar trend as for the compressive modulus: bulk GelMA had the highest modulus, in the order of G′˜30 kPa and compressive modulus˜200 kPa, and the 3D bioprinted scaffold had the lowest due to the abundant interconnected void spaces and filament spacing, with G′˜10 kPa and compressive modulus˜20 kPa. FIG. 16G shows loss tangent values at the same strain rate (0.1%) and frequency (1 rad/s), comparing loss and storage moduli of samples, which implies that all scaffolds have solid-like behavior as their storage modulus is 1 to 2 orders of magnitude larger than the loss modulus.

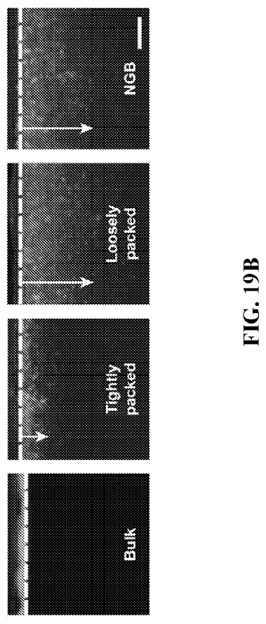

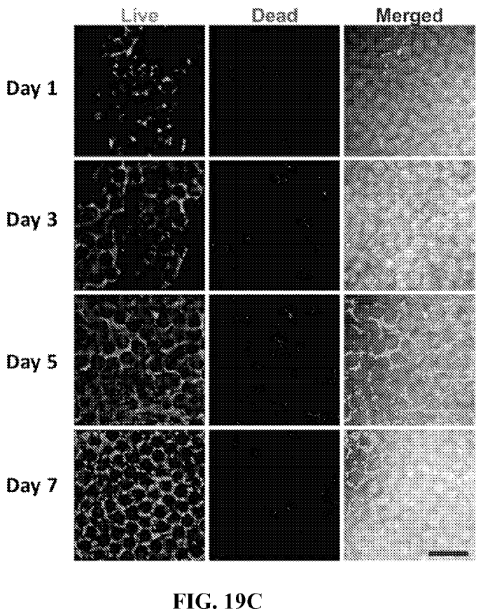

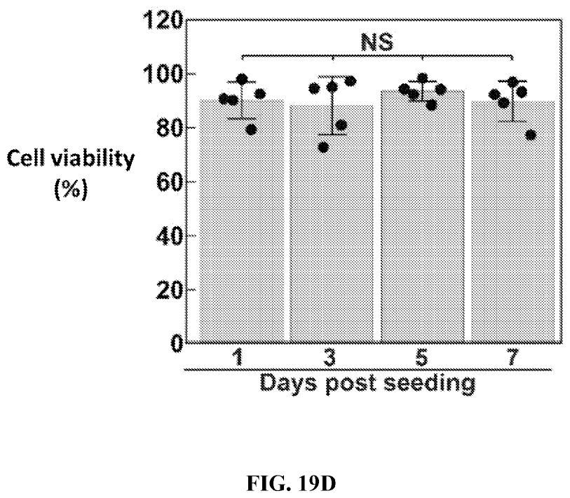

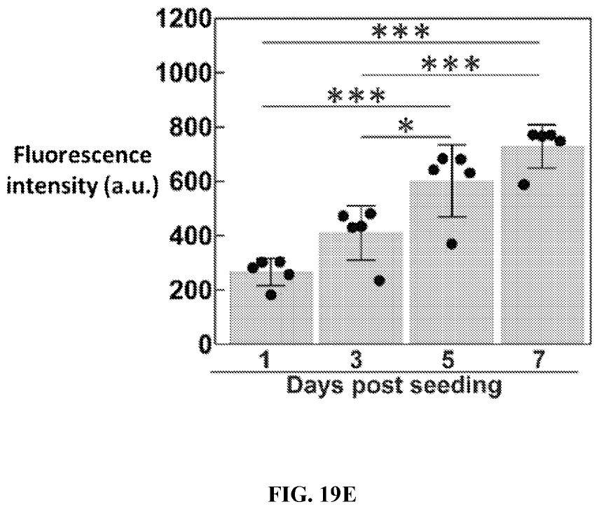

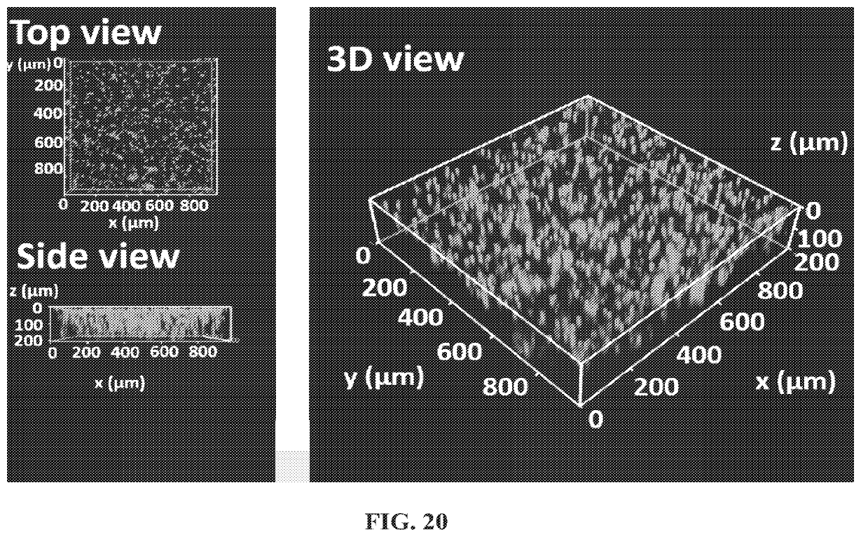

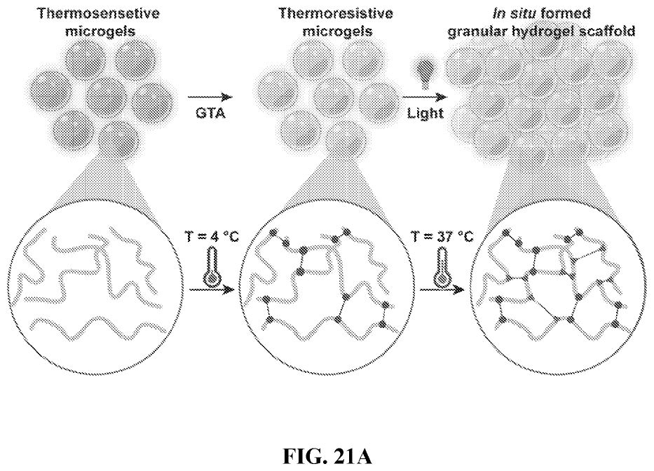

In vitro analyses were performed on 3D bioprinted NGB scaffolds to assess post-printing, on-demand cell seeding and penetration, metabolic activity, and viability. FIG. 19A schematically shows the topical NIH/3T3 murine fibroblast cell seeding in the 3D bioprinted NGB scaffolds, followed by cell penetration under gravitational and capillary forces originated from the microscale interstitial void spaces. FIG. 20 shows the immediate penetration of cells after topical seeding on the bioprinted NGB scaffolds based on 3D z-stacked fluorescence images acquired within a 200 μm depth after 5 min. The NGB permits immediate cell penetration as a result of interconnected microporosity. Cells can readily penetrate in loosely packed microporous scaffolds depending on the pore size distribution and cell dimensions. To further analyze the penetration of cells into the scaffolds, fluorescently labeled cells were topically seeded and incubated for 4 h. The preserved microporosity of NGB enables the cells to immediately penetrate the 3D bioprinted scaffolds, similar to a loosely packed scaffold.

FIG. 19B shows cross-sectional fluorescent images acquired using a low magnification fluorescence microscope (total magnification of 40×) for conventional bulk, loosely packed, tightly packed, and NGB scaffolds. The nanoporous bulk GelMA did not allow cell penetration, and all cells remain on top of it. The tightly packed scaffolds underwent limited cell penetration, typically around 400% less penetration depth than in the loosely packed and NGB scaffolds. Cell penetration into granular hydrogels is regulated by pore size distribution and interconnectivity, which is governed by hydrogel particle size and packing condition. Using different hydrogel particle shapes and packing densities, the infiltration of cells into granular scaffolds has been studied both in vitro and in vivo. The more packed the hydrogel particles, the more difficult it is for cells to penetrate. Overall, NGB enables facile cell penetration without requiring any porogen and/or sacrificial components, similar to the conventional loosely packed microgel scaffolds.

FIG. 19C shows live/dead staining of topically seeded NGB scaffolds over 7 days. The cells readily adhere to and proliferate among the hydrogel particles, in the interstitial void spaces. FIG. 19D presents the quantification of live/dead images based on normalizing the number of live cells (stained in green) to the total numbers of cells. According to this figure, more than 90% of cell remain viable within the post-seeding course of culture. In addition, the PrestoBlue™ assay showed ˜3- and ˜5-fold increase in the metabolic activity of cells on days 5 and 7, respectively, as presented in FIG. 19E. Such a high cell viability in the 3D bioprinted NGB scaffolds is a result of facile topical cell seeding, interconnected microporosity, and enhanced oxygen and metabolite transport. It is important to note that previous work has shown stiff bulk hydrogels, e.g., bulk GelMA (20% w/v), completely compromise the viability of encapsulated fibroblast cells (cell viability˜0%). Accordingly, the NGB provides the first class of granular bioinks that enables post-bioprinting 3D topical seeding, which may eliminate the necessity of bioorthogonality for developing 3D cell-laden scaffolds. This technology may be expanded to other hydrogel particle platforms made up of synthetic, natural, or hybrid polymeric hydrogel particles, which may be assembled to each other using similar nanoparticles or other physical and/or chemical methods, such as charge-induced reversible binding, host-guest interactions, or dynamic covalent bonds, among others.

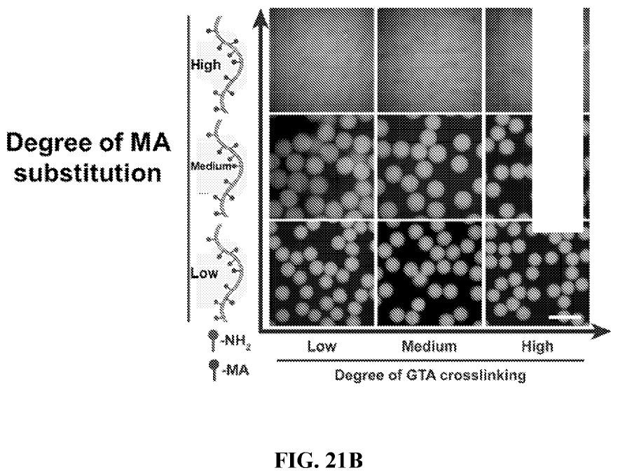

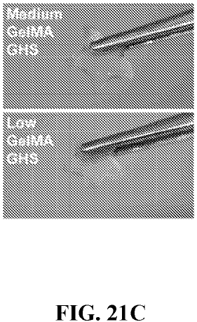

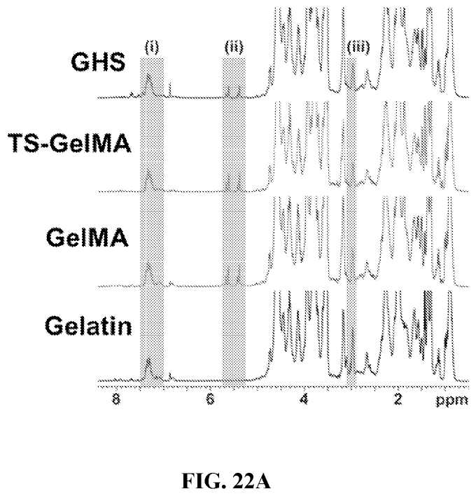

FIGS. 21A-C show in situ formation of granular hydrogel scaffolds (GHS) from thermoresistive GelMA microgels. FIG. 21A shows that GelMA hydrogel particles are thermoresponsive and unable to form a scaffold at body temperature (e.g., 37° C.). To overcome this limitation, the GelMA biopolymer was crosslinked via a Schiff-base reaction to produce thermoresistant microgels. These thermoresistive microgels served as building blocks for the in situ fabrication of GHS via radical photopolymerization.

FIG. 21B shows that GelMA was synthesized using three different degrees of substitutions (DOS) with high (˜70%), medium (˜40%) and low (˜10%), which affected the availability of primary amine groups for Schiff-base crosslinking using glutaraldehyde (GTA). High DoS resulted in insufficient primary amine groups, making the thermoresistive microgel unstable. In contrast, medium and low DoS resulted in stable thermoresistive microgels.

FIG. 21C shows thermoresistive microgels from medium and low GelMA were scaffolded to form a mechanically stable GHS.

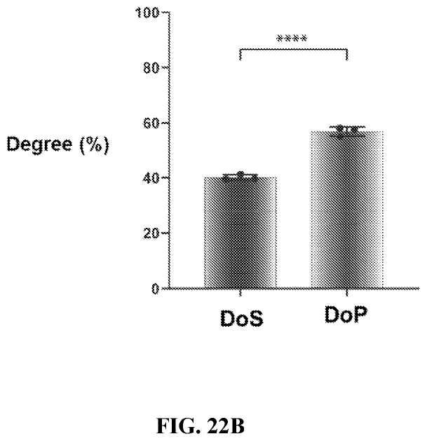

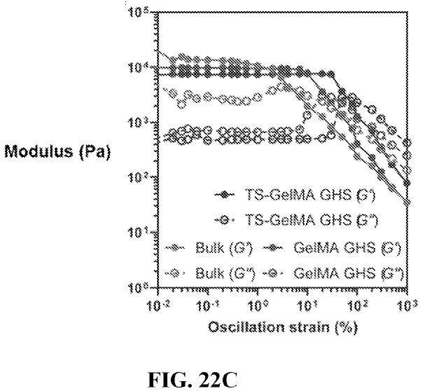

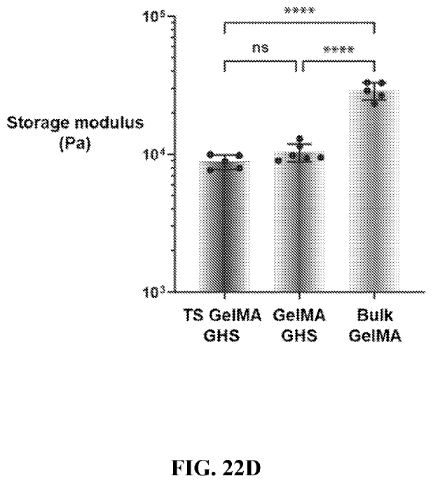

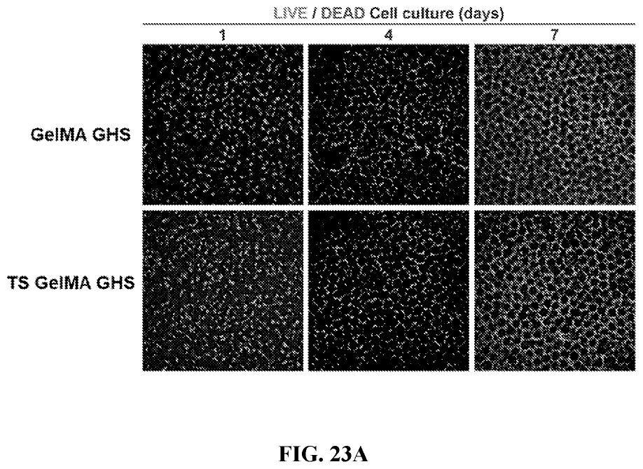

FIGS. 22A-D show material and mechanical characterization of GHS made from thermoresistant microgels. FIG. 22A shows ¹H NMR spectrometry of pure gelatin microgel, uncrosslinked GelMA microgel (with medium DoS), thermoresistant GelMA hydrogel particles, and photocrosslinked GHS composed of thermoresistant microgels. The peaks for aromatic acids (i) serve as the reference in all the groups. The presence of vinyl groups (ii) and a decrease in lysine protons (iii) were also observed.

FIG. 22B shows oscillatory strain sweep at constant frequency of 1 rad/s performed on in situ fabricated scaffolds: GHS from thermoresistant GelMA microgels (TS GelMA GHS), GHS from thermoresponsive GelMA microgels (GelMA GHS), and conventional (bulk) GelMA scaffolds.

FIG. 22C shows dynamic moduli versus oscillation strain for different scaffolds at a constant frequency of 1 rad/s, showing the storage and loss moduli of TS GelMA GHS, GelMA GHS, and conventional (bulk) GelMA scaffolds.

FIG. 22D shows the storage modulus measured at 1 rad/s and 0.1% strain. No significant difference was observed between the TS GelMA GHS and GelMA GHS, but bulk GelMA had significantly higher stiffness.

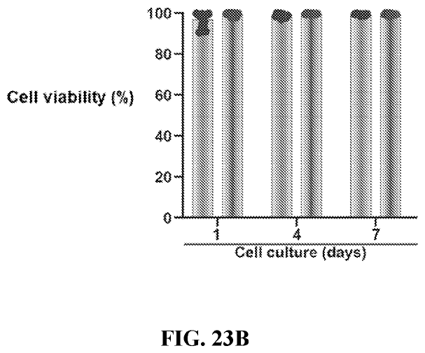

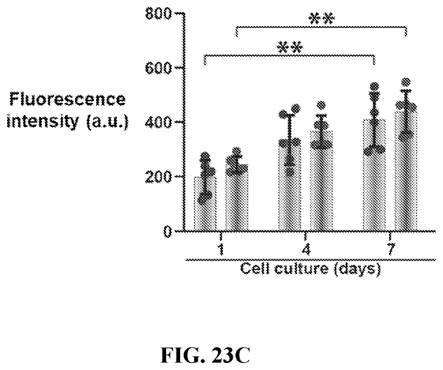

FIGS. 23A-C show biocompatibility assessment of GHS made from thermoresistant microgels. FIG. 23A shows that NIH-3T3 murine fibroblast cells were mixed with thermoresponsive or thermoresistant microgels, then photocrosslinked to form GelMA GHS or TS GelMA GHS, respectively. Fluorescent microscopy using a combination of green (representing live cells) and red (representing dead cells) dyes was performed over a period of 7 days, demonstrating cell proliferation and viability.

FIG. 23B shows the results if cell viability percentage indicated a consistently high viability rate (˜100%) for all GHS samples.

Overall, granular bioinks with well-preserved microscale porosity for the 3D extrusion bioprinting of tissue engineering scaffolds have been developed. Suspensions of otherwise non-extrudable loosely packed hydrogel particles have been rendered 3D bioprintable (NGB) via engineering the interparticle interactions based on the reversible interfacial self-assembly of heterogeneously charged nanoparticles. The NGB attains a shear-yielding behavior with prompt recovery, yielding higher hanging filament length, i.e., higher shape fidelity compared with tightly packed granular hydrogels. In addition, the NGB enables the sub-mm resolution extrusion bioprinting of granular scaffolds with cell-scale interconnected microporosity, ready to be 3D seeded with cells after printing. Considering the emerging interests of granular hydrogels for in situ tissue engineering, regeneration, and modeling, it is speculated that the unique, unprecedented properties of NGB, such as preserved porosity and maximized printability and shape fidelity, may enable a novel universal platform for the biofabrication of granular scaffolds.

### Experimental Section

### Microfluidic Device Fabrication

High-throughput step emulsification microfluidic devices were fabricated via soft lithography in the Nanofabrication facilities of Materials Research Institute (MRI) at the Pennsylvania State University (Penn State) according to an established protocol. Master molds were fabricated using KMPR® 1000 series (Kayaku Advanced Materials, MA, USA) as negative photoresists on 4-inch mechanical grade silicon wafers (UniversityWafer, MA, USA). A Two-layer lithography process was performed with KMPR® 1025 and KMPR 1035 for the first and second layers, respectively. UV light exposure and alignment were performed by MA/BA6 (SÜSS MicroTeck, Germany) using designed alignment marks. UV exposure dose was set to 645 and 2000 mJ/cm² for the first and second layers, respectively. Soft bake and post bake durations were adopted from the photoresist technical datasheet. The thickness of the first (27 μm) and second (140 μm) layers regulates the nozzle and reservoir height, respectively, which was measured using a profilometer (Tecncor P16+, KLA, CA, USA). The devices were molded on the masters using the polydimethylsiloxane (PDMS) Sylgard 184 kit (Dow Corning, MI, USA). The base and crosslinker were well-mixed at a 10:1 mass ratio, vacuum degassed, poured onto the masters, vacuum degassed again to remove all the air bubbles, and cured at 80° C. for 2 h. The PDMS devices were cut, plasma bonded to glass microscope slides (VWR, PA, USA), and placed in the oven to facilitate the bonding at 80° C. for 2 h.

### GelMA Synthesis

GelMA was synthesized according to the following method. Gelatin (20 g, Type A from porcine skin, Sigma, MA, USA) was dissolved in 200 mL of Dulbecco's phosphate-buffered saline (DPBS) at 50° C. while stirring at 200 rpm. Then, 16 mL of methacrylic anhydride (MAA, Sigma, MA, USA) was added dropwise to the stirring solution at 50° C., and the reaction beaker was wrapped in aluminum foil to protect from light. After 2 h, the reaction was stopped by adding 400 mL of excess DPBS. The solution was dialyzed against ultra-pure Milli-Q® water (electrical resistivity≈18 MΩ at 25° C., Millipore Corporation, MA, USA) for 10 days at 40° C. using 12-14 kDa molecular weight cutoff (MWCO) membranes (Spectrum Laboratories, NJ, USA) to remove unreacted MAA. A clear solution was obtained, sterile filtered using a 0.20 μm vacuum filtration unit (VWR, PA, USA), and frozen at −80° C., followed by lyophilization to yield white GelMA solid.

### Proton Nuclear Magnetic Resonance (1H NMR) Spectroscopy

The degree of methacrylate/methacryloyl substitution (DS) of GelMA was determined using 1H NMR (400 MHz Bruker NEO, MA, USA) at the NMR facilities of Penn State according to an established method. About 40 mg of gelatin powder and lyophilized GelMA were separately dissolved in 2 mL of deuterium oxide (D₂O, Sigma, MO, USA) for 2 h at 37° C. The area was integrated using TopSpin 4.0.7 software considering the peaks of aromatic acids, which appeared at a chemical shift around 6.5-7.5 ppm as the reference. The lysine methylene proton peaks at a chemical shift around 3.0 ppm were used to determine the DS according to the following equation:

$$DS ( \% ) = ( 1 - \frac{Area of lysine methylene in GelMA}{Area of lysine methlene in gelatin} ) \times 100$$

### Microgel Fabrication

GelMA polymer was dissolved in Dulbecco's modified eagle medium (DMEM) or DPBS, including 0.1% w/v PI (lithium phenyl-2,4,6-trimethylbenzoylphosphinate, LAP, Sigma, MO, USA) at 50° C. to prepare a GelMA solution (10% w/v). The GelMA solution was used as the dispersed (aqueous) phase, and a mixture of Novec 7500™ engineering oil (3M, MN, USA), containing 2% v/v of a surfactant (Pico-Surf™, Sphere Fluidics, Cambridge, UK), was used as the continuous (oil) phase. The solutions were loaded into 5 mL syringes (BD, NJ, USA) and injected into the device using syringe pumps (PHD 2000, Harvard Apparatus, MA, USA) to form an emulsion of GelMA droplets in the oil. The droplet fabrication system was maintained at 35-40° C. using a space heater. The droplet suspension in oil was collected and stored at 4° C. overnight while protecting from light to obtain physically crosslinked GelMA microgels.

### Preparation of Bulk GelMA Hydrogel Scaffold

To prepare bulk (nonporous) hydrogel scaffolds, lyophilized GelMA was dissolved in DPBS containing 0.1% w/v of the PI at 50° C. to yield a clear GelMA solution (10% w/v). The GelMA solution was pipetted in laser-cut cylindrical acrylic molds with pre-warmed (50° C.) pipette tips (VWR, PA, USA), and place in a custom-built dark humidity chamber. The humidity chamber was a petri dish (100 mm, VWR, PA, USA) filled with wet paper towels to prevent drying, wrapped in aluminum foil to avoid light exposure. The molded GelMA solution was physically crosslinked inside the humidity chamber at 4° C. for 8 h, following by chemical photocrosslinking using light with a wavelength of 395-405 nm and intensity of 15 mW cm-2 for 60 s.

### Preparation of Granular Hydrogel Scaffolds

GelMA microgels in the oil were washed with 1H, 1H,2H,2H-perfluoro-1-octanol (PFO, 20% v/v in Novec 7500™ oil, Alfa Aesar, MA, USA) at a 1:1 volume ratio and centrifuged at 300×g for 15 s (including the ramp-up) to remove the surfactant and oil from the suspension. A PI solution (0.1% w/v in DPBS) was added to the washed microgel suspension and vortexed immediately for 5 s. Then, the suspension was centrifuged once at 2,940×g for 15 s or five times at 16,000×g for 60 s to yield loosely packed and tightly packed granular systems, respectively. Loosely packed microgel suspensions were pipetted out using a positive displacement pipette (Microman E M100E, Gilson, OH, USA) and injected into a cylindrical laser-cut acrylic mold (diameter=8 mm, height=3 mm). Tightly packed microgels were scooped out using a spatula and molded similarly. Then, the hydrogels were photocrosslinked via light exposure (wavelength=395-405 nm and intensity=15 mW cm⁻²) for 60 s to form microgel-microgel covalent bonds as a result of the free radical polymerization of GelMA vinyl groups.

### Colloidal Gel Preparation

To make the colloidal gel, silicate nanoparticles (LAPONITE® XLG nanoplatelets, BYK USA Inc., CT, USA) were exfoliated in cold Milli-Q® water at 4° C. Briefly, a nanoparticle dispersion (3% w/v) was prepared by adding 100 mg f LAPONITE® powder to 3.333 mL of Milli-Q® water at 4° C., followed by immediate vigorous vortexing for at least 15 min until a clear dispersion was obtained. The dispersion was maintained at 4° C. for at least 24 h to obtain aging-induced colloidal hydrogels.

### NGB Preparation

A silicate nanoparticle dispersion (3.33% w/v) was prepared by adding 100 mg of LAPONITE® powder to 3 mL of Milli-Q® water, followed by exfoliation until a clear dispersion was obtained. From a stock solution of PI (LAP, 1.0% w/v in Milli-Q® water at 4° C.), 333 μL was added to the nanoparticle dispersion to obtain 3% w/v nanoparticle dispersion containing 0.1% w/v of the PI. In parallel, physically crosslinked GelMA microgel suspension in oil were washed with PFO (20% v/v in Novec 7500™ oil) at a 1:1 volume ratio and centrifuged at 300×g for 15 s (including the ramp-up) to remove the surfactant and oil from the suspension at 4° C. The silicate nanoparticle dispersion was then added to the microgel suspension and vortexed immediately for 15 s to ensure that the microgels and nanoparticles were well mixed. The resulting suspension was then centrifuged at 2,940×g for 15 s (including ramp-up), the supernatant was removed, and the microgel nanocomposite was aged for 1 day at 4° C. to form the NGB. The NGB was loaded into Luer-Lock syringes (3 mL, BD, NJ, USA), pulse centrifuged at 300×g to remove trapped air, and maintained at 4° C. while covered with aluminum foil to protect from light for further analyses or 3D bioprinting.

### Rheological Assessments of NGB

To characterize the rheological properties of NGB, an AR-G2 Rheometer (TA instrument, DE, USA) equipped with parallel plates (upper plate diameter=20 mm and truncation gap=1000 μm). The lower plate temperature was set to 4° C., the NGB was loaded on it, sandwiched by the upper plate, and its excess amount was trimmed. The rheological behavior of NGB was characterized by several tests, including: (i) steady shear rate sweep, where the shear rate was altered from 10⁻² to 10² s⁻¹, (ii) oscillatory strain sweep at strain of 10⁻² to 5×10²% and a constant oscillation frequency of 1 rad s⁻¹, (iii) oscillatory frequency sweep at 10⁻² to 10² rad s⁻¹ and constant strain of 0.1%, (iv) a recovery test, performed by alternating the strain between 1% and 500% over time at a constant frequency of 1 rad s⁻¹, and (v) time sweep, which was performed on freshly prepared (non-aged) NGB at a constant frequency of 1 rad s⁻¹ and strain of 0.1% over time for 4 h.

### Molecular Transport Assessment

To assess the pore availability and interconnectivity, bulk (nanoporous) and granular hydrogels were examined. Bulk GelMA (10% w/v) was prepared in DPBS containing 0.1% PI at 37° C. Colloidal gel, tightly packed, and loosely packed systems were prepared as previously described. All materials except the bioink were casted in a cylinder with a diameter of 8 mm and height of 3 mm. The NGB was 3D bioprinted in a similar shape. Samples were placed in a custom-developed PDMS mold (FIG. 9), followed by adding 10 μL of high molecular weight (average molecular weight˜2 MDa) fluorescein isothiocyanate-dextran (FITC-dextran, Sigma, MO, USA) solution with a concentration of 30 μM. FITC-dextran diffusion in the samples was imaged over time using the Leica DMi8 THUNDERED™ microscope (Germany).

### Characterization of Scaffold Mechanical Properties

Compression tests were performed on the hydrogel scaffolds photocrosslinked as cylindrical specimen (diameter=8 mm, height=1 mm) using the Instron mechanical tester (Instron 5542, MA, USA). The compression rate was 1 mm min⁻¹, and stress versus strain curves were obtained. The best linear fit at the elastic region (strain˜5%-15%) was obtained, and the slope was reported as the compressive modulus. For viscoelasticity analysis, cylindrical hydrogel samples (diameter=8 mm, height=1 mm) were prepared. Oscillatory shear rheology was performed using the rheometer at 25° C. The top and bottom plates were 8 mm and 25 mm, respectively. The storage and loss moduli were recorded first based on amplitude sweep for furnishing linear viscoelastic region (LVR) at a fixed frequency (1 rad s⁻¹) from 0.1 to 100% of strain, and then 637 based on frequency sweep at 0.1% of strain (in the LVR) from 0.1 to 100 rad s⁻¹.

### Characterization of Maximum Hanging Filament Length

To measure the maximum hanging filament length, mechanical extrusion of NGB, tightly packed, and loosely packed granular hydrogels was conducted using the Instron mechanical tester (Instron 5542, MA, USA). Samples were loaded in 3 mL syringes (BD, NJ, USA). Blue (22G), red (25G), and clear (27G) nozzles with an inner diameter of 410, 250, and 200 μm, respectively, were used. The injection rate was 4.25 mm min-1 (flow rate=250 AL min⁻¹). Extruded filaments were recorded using a Canon SX700HS camera and analyzed using the ImageJ software (FIJI, version 1.53n, NIH, MD, USA)

### Porosity Characterization

Molded and 3D bioprinted hydrogel samples were incubated in a high molecular weight FITC-dextran solution (Mw˜2 MDa, 15 μM in Milli-Q® water) for 30 min to fill the void spaces among the microgels with the dye. Pore size distribution and void fraction were analyzed using 3D z-stacked images acquired using the Leica DMi8 THUNDER™ microscope. One hundred z-stacks were captured for each sample, covering a total depth of 140 μm. The void fraction was measured using the built-in microscope software (LAS X 5.0.3 Life Science Microscope Software Platform) based on the ratio of stained interstitial space over total volume. Microporosity was characterized using a custom-built computer code (MATLAB, version 2021b) as previously described. For the median pore diameter, the equivalent diameter of detected pores was measured based on circles of similar area, followed by calculating the median value for each sample.

### Three-dimensional (3D) Bioprinting

NGB was 3D bioprinted using a BIO X bioprinter (Cellink, MA, USA). To conduct bioprinting, the bioink was loaded in 3 mL cartridges (Cellink, MA, USA) using a female-female Luer-Lok™ connector, pulse centrifuged at 200×g to eliminate air bubbles, wrapped in aluminum foil to protect from light, and maintained at 4° C. The printing bed temperature was set to 4° C., and the printed structures were exposed to light (wavelength=395-405 nm, intensity=15 mW/cm²) for up to 4 min, depending on the sample thickness.

### Printability Assessment

For printability quantification, two consecutive layers of NGB were printed on top of each other. Printed constructs followed horizontal or vertical (0 or) 90° printing of 15 mm parallel lines pattern with increasing distance between adjacent filaments according to an established method. The printing window was deposited using a custom-developed G-code file, ranging from 1×1 to 5×5 mm² with 1 mm increments. Digital single-lens reflex (DSLR) and tile scan microscopic images of printed constructs were taken right after the fabrication by a Canon SX700HS camera and Leica DMi8 microscope, respectively. The images are analyzed using ImageJ software by inverting, thresholding, and analyzing particles (FIG. 13). The printability (Pᵣ) was measured using the below equation, where L denotes the actual perimeter of the printing window (μm), and A is the actual area (μm²).

$${P}_{r} = \frac{{L}^{2}}{16 \times A}$$

### Shape Fidelity Analysis

The standard shape fidelity test was performed by 3D bioprinting a hollow cylindrical structure with a diameter of 5 mm and height of 5 or 10 mm. The deposited layer numbers ranged from 12 to 50 depending on the filament size and the cylinder height. For the 5 mm high cylinder, the number of layers were 12, 20, and 25 for the 22G, 25G, and 27G nozzles, respectively. In addition, for the longer, 10 mm high cylinder, the number of layers were 24, 40, and 50 for the 22G, 25G, and 27G nozzles, respectively. The DSLR images of 3D bioprinted structures were captured by a Canon SX700HS camera, and measurements were performed using the ImageJ software. The height collapse factor was measured as the ratio of actual (Hₐ) height to the theoretical (Hₜ) height, as described in the following equation:

$${H}_{f} = \frac{{H}_{a}}{{H}_{t}}$$

### In Vitro Cell Culture

NIH/3T3 murine fibroblast cells (ATCC, VA, USA) were cultured in DMEM, supplemented with 10% v/v fetal bovine serum and 1% v/v antibiotic. The media was refreshed every other day, and the cells were passaged when they reached 80% of confluency, typically twice per week. A standard cell culture incubator (Eppendorf, Hamburg, Germany) was used to culture cells in T-75 cell culture flasks (VWR, PA, USA) under a 5% v/v CO₂ atmosphere at 37° C. Fibroblasts were trypsinized using 0.25% trypsin-EDTA (Sigma, MO, USA), followed by cell counting using an automated cell counter device (Countess 2, Thermo Fisher Scientific, MA, USA) and resuspension in the media for 3D topical cell seeding.

### Topical Cell Seeding in 3D Bioprinted NGB Scaffolds

Topical cell seeding in the 3D bioprinted NGB scaffolds was performed to evaluate cell penetration and viability. The NGB scaffolds were 3D bioprinted and soaked in DPBS supplemented with 1% v/v antibiotic overnight. Then the scaffolds were placed in a non-treated 48-well cell culture plate (Cellstar®, Greiner Bio-One, Austria) filled with DBPS. DPBS was then discarded, and 20 μL of the cell suspension containing 5 million cells per mL was added on top of each scaffold. The scaffolds were incubated for 30 min to ensure cell-scaffold adhesion, followed by adding the complete cell culture media. The seeded scaffolds were incubated, and the media was refreshed daily.

### Metabolic Activity Assessment

PrestoBlue™ cell viability kit (Invitrogen, MA, USA) was used to evaluate the cellular metabolic activity on days 1, 4, and 7 based on the manufacturer protocol. PrestoBlue was mixed with DMEM in a 1:10 v/v ratio to make a 1× solution. For each cell-seeded scaffold, 500 μL of PrestoBlue 1× solution was added to the well and incubated for 4 h at 37° C. The supernatant was removed and poured into 96-well plates (Cellstar®, Greiner Bio-One, Austria). Fluorescence intensity was recorded using a microplate reader (Tecan Infinite M Plex, Männedorf, Switzerland) at 530 nm excitation and 590 nm emission and corrected with respect to the background signal of the virgin PrestoBlue media.

### Cell Viability Assessment

Two-color fluorescence Live/Dead cell viability assay kit (Invitrogen, MA, USA) was used according to the manufacturer protocol to evaluate the cell viability of seeded scaffolds. Calcein AM (1 mL, 1 μM) was used for staining live cells, while BOBO™-3 Iodide (1 μL) was used as a dead cell indicator. The samples were incubated at 20° C. for 15 minutes and imaged using the Leica DMi8 fluorescent microscope. The live channel was set at 470 nm (blue) excitation and 510 nm (green) emission wavelengths. The dead channel was set to 550 nm (green) excitation and 610 nm (red) emission wavelengths.

### Statistical Analysis

Data was acquired with at least three iterations. For the analysis of significance, the one-way analysis of variance (ANOVA) test was carried out, followed by the Tukey and Bonferroni post hoc tests. Groups were considered significantly different if the p-value was lower than 0.05. The level of significance was noted with *p<0.05, **p<0.01, and *** p<0.001.

### REFERENCES

Each of the following references is incorporated herein by reference in its entirety.
[1] S. Derakhshanfar, R. Mbeleck, K. Xu, X. Zhang, W. Zhong, M. Xing, Bioact. Mater. 2018, 3,
[2] S. M. Hull, L. G. Brunel, S. C. Heilshorn, Adv. Mater. 2022, 34, 1.
[3] Y. S. Zhang, G. Haghiashtiani, T. Hübscher, D. J. Kelly, J. M. Lee, M. Lutolf, M. C. McAlpine, W. Y. Yeong, M. Zenobi-Wong, J. Malda, Nat. Rev. Methods Prim. 2021, 1, 75.
[4] N. Annabi, A. Tamayol, J. A. Uquillas, M. Akbari, L. E. Bertassoni, C. Cha, G. Camci-Unal, M. R. Dokmeci, N. A. Peppas, A. Khademhosseini, Adv. Mater 2014, 26, 85.
[5] Y. S. Zhang, A. Khademhosseini, Science. 2017, 356, caaf3627.
[6] C. F. Guimarães, L. Gasperini, A. P. Marques, R. L. Reis, Nat. Rev. Mater. 2020, 5, 351.
[7] P. G. de Gennes, T. A. Witten, Phys. Today 1980, 33, 51.
[8] N. Annabi, J. W. Nichol, X. Zhong, C. Ji, S. Koshy, A. Khademhosseini, F. Dehghani, Tissue Eng.—Part B Rev. 2010, 16, 371.
[9] K. J. De France, F. Xu, T. Hoare, Adv. Healthc. Mater. 2018, 7, 1700927.
[10] J. Malda, J. Visser, F. P. Melchels, T. Jüngst, W. E. Hennink, W. J. A. Dhert, J. Groll, D. W. Hutmacher, Adv. Mater. 2013, 25, 5011.
[11] R. Levato, T. Jungst, R. G. Scheuring, T. Blunk, J. Groll, J. Malda, Adv. Mater. 2020, 32, 1906423.
[12] W. Sun, B. Starly, A. C. Daly, J. A. Burdick, J. Groll, G. Skeldon, W. Shu, Y. Sakai, M. Shinohara, M. Nishikawa, J. Jang, D. W. Cho, M. Nie, S. Takeuchi, S. Ostrovidov, A. Khademhosseini, R. D. Kamm, V. Mironov, L. Moroni, I. T. Ozbolat, Biofabrication 2020, 12, 22002.
[13] Q. Feng, D. Li, Q. Li, X. Cao, H. Dong, Bioact. Mater. 2022, 9, 105.
[14] A. C. Daly, L. Riley, T. Segura, J. A. Burdick, Nat. Rev. Mater. 2020, 5, 20
[15] D. R. Griffin, W. M. Weaver, P. O. Scumpia, D. Di Carlo, T. Segura, Nat. Mater. 2015, 14, 737.
[16] D. R. Griffin, M. M. Archang, C.-H. Kuan, W. M. Weaver, J. S. Weinstein, A. C. Feng, A. Ruccia, E. Sideris, V. Ragkousis, J. Koh, M. V. Plikus, D. Di Carlo, T. Segura, P. O. Scumpia, Nat. Mater. 2021, 20, 560.
[17] L. R. Nih, E. Sideris, S. T. Carmichael, T. Segura, Adv. Mater. 2017, 29, 1606471.
[18] L. R. Nih, S. Gojgini, S. T. Carmichael, T. Segura, Nat. Mater. 2018, 17, 642.
[19] J. Fang, J. Koh, Q. Fang, H. Qiu, M. M. Archang, M. M. Hasani-Sadrabadi, H. Miwa, X. Zhong, R. Sievers, D. W. Gao, R. Lee, D. Di Carlo, S. Li, Adv. Funct. Mater. 2020, 30, 2004307.
[20] C. B. Highley, K. H. Song, A. C. Daly, J. A. Burdick, Adv. Sci. 2019, 6, 1801076.
[21] S. Xin, D. Chimene, J. E. Garza, A. K. Gaharwar, D. L. Alge, Biomater. Sci. 2019, 7, 1179.
[22] B. N. Pfaff, L. J. Pruett, N. J. Cornell, J. de Rutte, D. Di Carlo, C. B. Highley, D. R. Griffin, ACS Biomater. Sci. Eng. 2021, 7, 422.
[23] H. Zhang, Y. Cong, A. R. Osi, Y. Zhou, F. Huang, R. P. Zaccaria, J. Chen, R. Wang, J. Fu, Adv. Funct. Mater. 2020, 30, 1910573.
[24] A. Ding, O. Jeon, D. Cleveland, K. L. Gasvoda, D. Wells, S. J. Lee, E. Alsberg, Adv. Mater. 2022, 2109394.
[25] Y. Fang, Y. Guo, M. Ji, B. Li, Y. Guo, J. Zhu, T. Zhang, Z. Xiong, Adv. Funct. Mater. 2021, 2109810.
[26] A. J. Seymour, S. Shin, S. C. Heilshorn, Adv. Healthc. Mater. 2021, 10, 2100644.
[27] K. Song, A. M. Compaan, W. Chai, Y. Huang, ACS Appl. Mater. Interfaces 2020, 12, 22453.
[28] M. Hirsch, A. Charlet, E. Amstad, Adv. Funct. Mater. 2021, 31, 2005929.
[29] S. Xin, K. A. Deo, J. Dai, N. K. R. Pandian, D. Chimene, R. M. Moebius, A. Jain, A. Han, A. K. Gaharwar, D. L. Alge, Sci. Adv. 2021, 7, eabk3087.
[30] F. da Cruz, S. Emam, M. Prochnow, J.-N. Roux, F. Chevoir, Phys. Rev. E 2005, 72, 021309.
[31] Y. Forterre, O. Pouliquen, Comptes Rendus Phys. 2018, 19, 271.
[32] L. Shao, Q. Gao, C. Xie, J. Fu, M. Xiang, Z. Liu, L. Xiang, Y. He, Bio-Design Manuf. 2020, 3, 30.
[33] M. Van Hecke, J. Phys. Condens. Matter 2010, 22, 33101.
[34] G. Biroli, Nat. Phys. 2007, 3, 222.
[35] R. P. Behringer, Comptes Rendus Phys. 2015, 16, 10.
[36] A. Baule, R. Mari, L. Bo, L. Portal, H. A. Makse, Nat. Commun. 2013, 4, 2194.
[37] H. M. Jaeger, S. R. Nagel, Science. 1992, 255, 1523.
[38] K. Yue, G. Trujillo-de Santiago, M. M. Alvarez, A. Tamayol, N. Annabi, A. Khademhosseini, Biomaterials 2015, 73, 254.
[39] A. I. Van Den Bulcke, B. Bogdanov, N. De Rooze, E. H. Schacht, M. Cornelissen, H. Berghmans, Biomacromolecules 2000, 1, 31.
[40] W. K. Kegel, H. N. W. Lekkerkerker, Nat. Mater. 2011, 10, 5.
[41] B. Ruzicka, E. Zaccarelli, L. Zulian, R. Angelini, M. Sztucki, A. Moussaïd, T. Narayanan, F. Sciortino, Nat. Mater. 2011, 10, 56.
[42] K. Song, D. Zhang, J. Yin, Y. Huang, Addit. Manuf. 2021, 41, 101963.
[43] Sigma-Aldrich, “FITC-labelled polysaccharides,” can be found under https://www.sigmaaldrich.com/technical-documents/articles/chemistry/fluorescently-labeled-dextrane.html, 2018.
[44] G. Gillispie, P. Prim, J. Copus, J. Fisher, A. G. Mikos, J. J. Yoo, A. Atala, S. J. Lee, Biofabrication 2020, 12, 022003.
[45] A. Habib, V. Sathish, S. Mallik, B. Khoda, Materials (Basel). 2018, 11, 454.
[46] K. Zhou, M. Dey, B. Ayan, Z. Zhang, V. Ozbolat, M. H. Kim, V. Khristov, I. T. Ozbolat, Biomed. Mater. 2021, 16, 045005.
[47] A. Schwab, R. Levato, M. D'Este, S. Piluso, D. Eglin, J. Malda, Chem. Rev. 2020, 120, 11028.
[48] T. Gao, G. J. Gillispie, J. S. Copus, A. P. R. Kumar, Y. J. Seol, A. Atala, J. J. Yoo, S. J. Lee, Biofabrication 2018, 10, 34106.
[49] B. Akhoundi, A. H. Behravesh, Exp. Mech. 2019, 59, 883.
[50] T. Yao, J. Ye, Z. Deng, K. Zhang, Y. Ma, H. Ouyang, Compos. Part B Eng. 2020, 188, 107894.
[51] H. Askanian, D. Muranaka de Lima, S. Commereuc, V. Verney, 3D Print. Addit. Manuf. 2018, 5, 319.
[52] J. An, J. E. M. Teoh, R. Suntornnond, C. K. Chua, Engineering 2015, 1, 261.
[53] A. Sheikhi, J. de Rutte, R. Haghniaz, O. Akouissi, A. Sohrabi, D. Di Carlo, A. Khademhosseini, Biomaterials 2019, 192, 560.
[54] T. H. Qazi, J. Wu, V. G. Muir, S. Weintraub, S. E. Gullbrand, D. Lee, D. Issadore, J. A. Burdick, Adv. Mater. 2022, 2109194.
[55] J. M. de Rutte, J. Koh, D. Di Carlo, Adv. Funct. Mater. 2019, 29, 1.
[56] K. Hosokawa, T. Fujii, I. Endo, Anal. Chem. 1999, 71, 4781.
[57] “KMPR® 1000 Kayaku Advanced Materials, Inc.,” can be found under https://kayakuam.com/products/kmpr-1000/, n.d.
[58] A. Sheikhi, J. de Rutte, R. Haghniaz, O. Akouissi, A. Sohrabi, D. Di Carlo, A. Khademhosseini, MethodsX 2019, 6, 1747.
[59] K. Yue, X. Li, K. Schrobback, A. Sheikhi, N. Annabi, J. Leijten, W. Zhang, Y. S. Zhang, D. W. Hutmacher, T. J. Klein, A. Khademhosseini, Biomaterials 2017, 139, 163.
[60] H. Stratesteffen, M. Köpf, F. Kreimendahl, A. Blaeser, S. Jockenhoevel, H. Fischer, Biofabrication 2017, 9, 045002.
[61] A. Sheikhi, S. Afewerki, R. Oklu, A. K. Gaharwar, A. Khademhosseini, Biomater. Sci. 2018, 6, 2073.
[62] A. A. Adib, A. Sheikhi, M. Shahhosseini, A. Simeunović, S. Wu, C. E. Castro, R. Zhao, A. Khademhosseini, D. J. Hoelzle, Biofabrication 2020, 12, 45006.
[63] “Fiji Downloads,” can be found under https://imagej.net/software/fiji/downloads, n.d.
[64] D. Chimene, K. K. Lennox, R. R. Kaunas, A. K. Gaharwar, Ann. Biomed. Eng. 2016, 44, 2090.
[65] L. Ouyang, J. P. Wojciechowski, J. Tang, Y. Guo, M. M. Stevens, Adv. Healthc. Mater 2022, U.S. Pat. Nos. 2,200,027, 2,200,027.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the apparatus and methods of using and making the same disclosed herein have been discussed and illustrated, it is to be distinctly understood that the invention is not limited thereto but may be otherwise variously embodied and practiced within the scope of the following claims.

What is claimed is:

1. A method of forming a three-dimensional hydrogel scaffold, the method comprising:
providing a granular bioink material comprising hydrogel particles, wherein the hydrogel particles are reversibly connected using a connection medium;
extruding the bioink material; and
exposing the extruded bioink material to a stimulus to form the three-dimensional hydrogel scaffold.

2. The method of claim 1, wherein the connection medium is nanoparticles.

3. The method of claim 1, wherein the hydrogel particles are selected from the group consisting of synthetic polymers, natural polymers, and/or semi natural polymers.

4. The method of claim 1, wherein the step of providing the granular bioink material comprises:
forming gelatin methacryloyl by combining gelatin and methacrylic anhydride;
forming the hydrogel particles by combining the gelatin methacryloyl and a photoinitiator; and
combining the hydrogel particles with a connection medium, wherein the connection medium is nanoparticles.

5. The method of claim 4, wherein the photoinitiator is selected from the group consisting of lithium phenyl-2,4,6-trimethylbenzoylphosphinate, Ruthenium, and Irgacure 2959.

6. The method of claim 1, wherein the three-dimensional hydrogel scaffold comprises granular filaments with a diameter less than 400 μm.

7. The method of claim 1, wherein the stimulus is a light source with a wavelength of 250-785 nm.

8. A granular bioink material suitable for providing a three-dimensional hydrogel scaffold comprising:
gelatin methacryloyl hydrogel particles; and
a connection medium configured to reversibly connect the hydrogel particles.

9. The bioink material of claim 8, wherein the connection medium is nanoparticles configured to be absorbed onto the hydrogel particles and wherein the nanoparticles are configured to allow for electrostatic bonding of the hydrogel particles.

10. The bioink material of 9, wherein the nanoparticles are silicate nanoparticles.

11. The bioink material of claim 10, wherein the nanoparticles are heterogeneously charged.

12. The bioink material of claim 9, wherein the hydrogel particles have a size between 1 nm-1 mm.

13. The bioink material of claim 9, wherein the hydrogel particles have an aspect ratio between 1-10.

14. A three-dimensional hydrogel scaffold comprising a plurality of granular filaments, each filament comprising gelatin methacryloyl hydrogel particles and optionally nanoparticles.

15. The three-dimensional hydrogel scaffold of claim 14, wherein the filaments have a diameter less than 400 μm.

16. The three-dimensional hydrogel scaffold of claim 14, wherein the scaffold has a mean pore size of 1-1000 μm.

17. The three-dimensional hydrogel scaffold of claim 14, wherein the scaffold has a mean pore size of 10-100 μm.

18. The three-dimensional hydrogel scaffold of claim 14, wherein the scaffold has a minimum pore size of 100 nm.

19. The three-dimensional hydrogel scaffold of claim 14, wherein the scaffold has a minimum pore size of 10 μm.

20. The three-dimensional hydrogel scaffold of claim 14, wherein the scaffold has a void fraction of 10-40%.
